(19) United States
(12) Patent Application Publication
(10) Pub. No.: US 2017/0285002 A1
(43) Pub. Date: Oct. 5, 2017

(54) METHOD FOR RECONSTITUTING TUMOR WITH MICROENVIRONMENT

(71) Applicant: Public University Corporation Yokohama City University, Yokohama-shi (JP)

(72) Inventors: Hideki TANIGUCHI, Yokohama-shi (JP); Yasuharu UENO, Yokohama-shi (JP); Takanori TAKEBE, Yokohama-shi (JP); Keisuke SEKINE, Yokohama-shi (JP); Ryo OKUDA, Yokohama-shi (JP)

(73) Assignee: Public University Corporation Yokohama City University, Yokohama-shi (JP)

(21) Appl. No.: 15/461,217

(22) Filed: Mar. 16, 2017

(30) Foreign Application Priority Data
Mar. 16, 2016 (JP) 2016-053074
Jan. 6, 2017 (JP) 2017-001445

(51) Int. Cl. G01N 33/50; A61K 35/39; G01N 21/64; C12N 5/00; C12Q 1/02

(52) U.S. Cl. CPC G01N 33/5011; C12N 5/0068; C12Q 1/02; G01N 21/64; A61K 35/39; A61K 35/13

(57) ABSTRACT

It is intended to develop a technique that can reproduce a microenvironment of cancer tissue and to construct a novel drug discovery screening system of high precision. It is also intended to provide a method for reconstituting human cancer tissue using primary human cancer cells that retain the properties of human tumor. The present invention provides a reconstituted cancer organoid reproducing a cancer microenvironment. The present invention also provides a method for preparing a cancer organoid from cancer tissue, a xenograft prepared from the cancer organoid, a method for preparing the xenograft, a method for evaluating treatment resistance of cancer, a method for evaluating invasion or metastasis of cancer, a method for evaluating recurrence of cancer, and a method for conducting prognostic prediction of cancer.

### TECHNICAL FIELD

The present invention relates to a reconstitution method for reproducing a microenvironment of human cancer tissue, and a use method thereof

### BACKGROUND ART

The development of novel methods for treating intractable cancers including pancreatic cancer is an urgent necessity. A tumor microenvironment constructed by the interaction between cancer cells and various cells present in the neighborhood of the cancer cells (e.g., mesenchymal cells such as tumor-related fibroblasts and vascular endothelial cells, and inflammatory cells such as macrophages) has been found to play an important role in the treatment resistance of cancer. For example, pancreatic cancer, a typical intractable cancer, is rich in stroma. It has been reported that: the tumor stroma of pancreatic cancer interferes with the penetration of anti-cancer drugs (Non Patent Literature 1: Cancer Cell. 20: 21 (3): 418-429, 2012); and a cytokine (IL-6) produced by the tumor stroma contributes to the apoptosis resistance of pancreatic cancer cells (Non Patent Literature 2: EMBO Mol Med. 1; 7 (6): 735-53, 2015). It has also been reported that: an immature tumor vascular network is responsible for poor drug delivery (Non Patent Literature 3: Cancer Cell. 10; 26 (5): 605-22, 2014); and Jagged 1 produced by vascular endothelial cells contributes to the anti-cancer drug resistance of cancer cells (Non Patent Literature 4: Cancer Cell. 17; 25 (3): 350-65, 2014). From these findings, the understanding of the tumor microenvironment and a reproduction method thereof are very important for the identification of therapeutic targets for cancer or drug discovery or development.

1) A method for transplanting a human cancer tissue fragment to an immunodeficient animal (method for preparing a cancer-bearing animal carrying human cancer tissue), 2) a method for reconstituting cancer tissue using an established cancer cell line, and 3) a method for reconstituting cancer tissue using primary cultured cancer cells derived from a cancer patient have so far been developed as approaches for artificially reconstituting human cancer tissue. However, the method 1) requires passaging the cancer tissue in the immunodeficient animal and therefore presents high cost problems. In addition, the possibility has been pointed out that during passage of tumor, properties are changed due to the invasion of mouse stroma cells. Also, it has been reported as to the method 2) that: unfavorable genetic and epigenetic changes in cancer cells occur during long-term culture of the cancer cells; and the constituents, other than the cancer cells, of a tumor microenvironment cannot be reproduced (Non Patent Literature 5: Nature Reviews Clinical Oncology, 9, 338-350, 2012; and Non Patent Literature 6: Oncology, 33, 1837-1843, 201). On the other hand, the method 3) circumvents the problems of the method 1) and the former problem of the method 2), but disadvantageously falls short of reproducing cancer stroma by existing culture methods (Non Patent Literature 7: Science, 324, 1457-1461, 2009). Owing to these problems, existing methods for evaluating cancer cells cannot reproduce a tumor or cancer microenvironment and cannot reproduce human cancer tissue.

### CITATION LIST

### Non Patent Literature

[Non Patent Literature 1] Cancer Cell. 20: 21 (3): 418-429, 2012

[Non Patent Literature 2] EMBO Mol Med. 1; 7 (6): 735-53, 2015

[Non Patent Literature 3] Cancer Cell. 10; 26 (5): 605-22, 2014

[Non Patent Literature 4] Cancer Cell. 17; 25 (3): 350-65, 2014

[Non Patent Literature 5] Nature Reviews Clinical Oncology, 9, 338-350, 2012

[Non Patent Literature 6] Oncology, 33, 1837-1843, 201

[Non Patent Literature 7] Science, 324, 1457-1461, 2009

### SUMMARY OF INVENTION

### Problems to be Solved by the Invention

An object of the present invention is to develop a technique that can reproduce a microenvironment of cancer tissue and to provide a method for reconstituting human cancer tissue.

### Means for Solving the Problems

The present inventors have reconstituted a human pancreatic cancer organoid by coculturing a human pancreatic cancer cell line (PANC-1, CFPAC-1, or SW1990), human vascular endothelial cells (human umbilical vein endothelial cells: HUVECs), and human mesenchymal cells (human mesenchymal stem cells: hMSCs). A pancreatic cancer xenograft having rich stroma and a ductal structure has been formed from this pancreatic cancer organoid. The present inventors have also reconstituted a duct-like structure or rich stroma in a human primary pancreatic cancer organoid by separating and culturing primary human pancreatic cancer cells from a clinical specimen of human pancreatic cancer and coculturing these cells with stromal cells (vascular endothelial cells and mesenchymal stem cells). Human pancreatic cancer tissue (pancreatic cancer xenograft) with a tumor microenvironment (having rich stroma or a ductal structure) has been formed from this human primary pancreatic cancer organoid. The reconstituted pancreatic cancer tissue with rich stroma has exhibited high anti-cancer drug resistance. On the basis of these findings, the present invention has been completed.

The present invention is summarized as follows:

(1) A reconstituted cancer organoid reproducing a cancer microenvironment.
(2) The cancer organoid according to (1), wherein the cancer microenvironment comprises cancer stroma.
(3) The cancer organoid according to (1) or (2), wherein the cancer organoid comprises cancer cells having the properties of epithelial cells.
(4) The cancer organoid according to any of (1) to (3) further reproducing a ductal structure.
(5) The reconstituted cancer organoid capable of reproducing at least one or more of treatment resistance, invasion or metastasis, and cancer recurrence.
(6) The cancer organoid according to (5), which has at least one or more of treatment resistance such as drug sensitivity, radiation sensitivity, immunotherapy sensitivity, and nutrition therapy sensitivity.
(7) A reconstituted cancer organoid allowing prognostic prediction of cancer.
(8) A method for preparing a cancer organoid, comprising: digesting cancer tissue in the presence of a proteolytic enzyme and a Rho kinase inhibitor and then obtaining an aggregate of cancer cells; passaging the aggregate and then separating the cancer cells; and coculturing the cancer cells with mesenchymal cells and vascular endothelial cells to form the cancer organoid.
(9) The method according to (8), wherein the cancer organoid reproduces a cancer microenvironment.
(10) The method according to (9), wherein the cancer microenvironment comprises cancer stroma.
(11) The method according to any of (8) to (10), wherein the cancer organoid comprises cancer cells having the properties of epithelial cells.
(12) The method according to any of (8) to (11), wherein the cancer organoid further reproduces a ductal structure.
(13) The method according to any of (8) to (12), wherein the cancer organoid reproduces at least one or more of treatment resistance, invasion or metastasis, and cancer recurrences.
(14) The method according to (13), wherein the treatment resistance of cancer is at least one or more of drug sensitivity, radiation sensitivity, immunotherapy sensitivity, and nutrition therapy sensitivity.
(15) The method according to any of (8) to (12), wherein the cancer organoid allows prognostic prediction of cancer.
(16) A method for preparing a xenograft reproducing a cancer microenvironment, comprising transplanting an animal with a reconstituted cancer organoid reproducing a cancer microenvironment.
(17) The method according to (16), wherein the cancer microenvironment of the xenograft comprises cancer stroma.
(18) The method according to (16) or (17), wherein the reconstituted cancer organoid comprises cancer cells having the properties of epithelial cells.
(19) The method according to any of (16) to (18), wherein the reconstituted cancer organoid further reproduces a ductal structure.
(20) The method according to any of (16) to (19), wherein the xenograft further reproduces a ductal structure.
(21) The method according to any of (16) to (20), wherein the xenograft reproduces at least one or more of treatment resistance, invasion or metastasis, and cancer recurrences.
(22) The method according to (21), wherein the treatment resistance of cancer is at least one or more of drug sensitivity, radiation sensitivity, immunotherapy sensitivity, and nutrition therapy sensitivity.
(23) The method according to any of (16) to (20), wherein the xenograft allows prognostic prediction of cancer.
(24) A xenograft reproducing a cancer microenvironment, the xenograft being obtained by transplanting a nonhuman animal with a reconstituted cancer organoid reproducing a cancer microenvironment.
(25) The xenograft according to (24), wherein the cancer microenvironment of the xenograft comprises cancer stroma.
(26) The xenograft according to (24) or (25), wherein the xenograft comprises cancer cells having the properties of epithelial cells.
(27) The xenograft according to any of (24) to (26) further reproducing a ductal structure.
(28) A reconstituted cancer organoid-derived xenograft reproducing at least one selected from one or more of treatment resistance, invasion or metastasis, and cancer recurrences.
(29) The xenograft according to (28), wherein the treatment resistance of cancer is at least one or more of drug sensitivity, radiation sensitivity, immunotherapy sensitivity, and nutrition therapy sensitivity.
(30) A reconstituted cancer organoid-derived xenograft allowing prognostic prediction of cancer.
(31) A reconstituted cancer organoid-derived xenograft reproducing expression of a drug transporter.
(32) A reconstituted cancer organoid-derived xenograft having tumor vessels.
(33) A reconstituted cancer organoid-derived xenograft reproducing drug leakage characteristic of tumor vessels.
(34) A method for evaluating treatment resistance of cancer using a cancer organoid according to any of (1) to (7) and/or a xenograft according to any of (24) to (33).
(35) A method for evaluating invasion or metastasis of cancer using a cancer organoid according to any of (1) to (7) and/or a xenograft according to any of (24) to (33).
(36) A method for evaluating recurrence of cancer using a cancer organoid according to any of (1) to (7) and/or a xenograft according to any of (24) to (33).
(37) A method for conducting prognostic prediction of cancer using a cancer organoid according to any of (1) to (7) and/or a xenograft according to any of (24) to (33).
(38) A nonhuman animal carrying a xenograft according to any of (24) to (33).

The present invention enables elucidation of the treatment resistance mechanism of human cancer and construction of a novel drug discovery screening system.

### Advantageous Effects of Invention

The cancer organoid and the xenograft of the present invention can reproduce a cancer microenvironment with cancer stroma. The cancer organoid and the xenograft of the present invention can also reproduce cancer tissue (e.g., a ductal structure) similar to a structure in patients. The xenograft having stroma reduces the drug sensitivity of cancer cells.

### BRIEF DESCRIPTION OF DRAWINGS

The patent or application file contains at least one color drawing. Copies of this patent or patent application publication with color drawing will be provided by the USPTO upon request and payment of the necessary fee.

FIG. 1 shows the in vitro (upper panels) and in vivo (lower panels) drug sensitivity to gemcitabine (GEM) of the established pancreatic cancer cell line. 8000 cells of human pancreatic cancer cell line were seeded in each well of a 96-well plate, and gemcitabine was added at 24 hours. At 72 hours, the number of viable cells was measured and summarized in a graph. The pancreatic cancer cells of each line were subcutaneously transplanted to NOD/Scid mice. Gemcitabine was intraperitoneally administered at 100 mg/kg from the point in time when the tumor volume reached 100 mm³. Change in tumor size after the gemcitabine administration was evaluated. The discrepancy between the in vitro anti-cancer drug sensitivity and the in vivo anti-cancer drug sensitivity was confirmed.

FIG. 2 shows a HE staining image of tumor collected from transplanted mice (upper panels). This figure also shows a pathological histology of patient with pancreatic cancer. In the group transplanted with human pancreatic cancer cells alone, a stromal-poor xenograft was formed. A ductal structure characteristic of pancreatic ductal adenocarcinoma is not found.

FIG. 3 shows the process of formation of a human iPS liver bud (left) and the process of formation of a human pancreatic cancer cell-derived organoid that is formed from a human pancreatic cancer cell line (e.g., CFPAC-1), HUVECs, and hMSCs.

FIG. 4 shows the time-lapse images of pancreatic cancer organoid reconstitution from a human pancreatic cancer cell line (e.g., CFPAC-1, PANC-1, or SW1990), HUVECs, and hMSCs. A cancer organoid was formed from any of pancreatic cancer cell line (e.g., CFPAC-1, PANC-1, or SW1990) (left diagrams). A cancer organoid was reconstituted from GFP-labeled HUVECs, Kusabira Orange (KO)-labeled hMSCs, and unlabeled cancer cells (right diagrams).

FIG. 5 shows the morphology of a cancer organoid formed from each human cancer cell line. The culture period is 1 day. Robust cell aggregation is observed in a group containing HUVECs and hMSCs.

FIG. 6 shows a tissue image of a xenograft formed after transplantation of a cancer organoid formed from each human cancer cell line. The left column shows a pathological histology of a patient with pancreatic ductal adenocarcinoma (PDAC) which is a typical pancreatic cancer. In the pancreatic ductal adenocarcinoma, highly fibrotic stroma is present around a ductal structure (constituted by EpCAM-positive cells). The presence of αSMA-positive cells (mesenchymal cells) is observed in the fibrotic region. Rich stroma (αSMA-positive cells) as well as a duct-like structure (constituted by EpCAM-positive cells) is observed in the pancreatic cancer organoid transplantation group. On the other hand, a pancreatic duct structure is not confirmed in a group transplanted with an aggregate prepared from established pancreatic cancer cells only (photographs of the middle column), and this group manifests structurally poor tissue.

FIG. 7 shows a tissue image of a xenograft formed by transplanting a human pancreatic cancer cell line-derived organoid to NOD/Scid mice (the first and second columns from the right of the left diagrams), a pancreatic cancer cell-alone transplantation group (the second column from the left of the left diagrams), and a tissue image of a primary lesion of human pancreatic cancer (leftmost column of the left diagrams). The rightmost column of the left diagrams shows a group transplanted with a cancer organoid rich in stromal cells. The second column from the right shows a group transplanted with a cancer organoid poor in stromal cells. A HE image, a CK7 and αSMA immunostaining image, a Sirius red staining image, an Azan staining image, and an Alcian blue staining image are shown in order from the upper to lower panels. Data on a quantified αSMA-positive ratio, Sirius red-positive area, and Azan stain-positive area are each shown on the right. From the HE staining image and the CK7 immunostaining image, the xenograft formed after human pancreatic cancer organoid transplantation is confirmed to manifest a ductal structure and stroma similar to the primary lesion of human pancreatic cancer as compared with the pancreatic cancer cell-alone transplantation group. By transplanting pancreatic cancer organoids with high frequency of stromal cells, a xenograft containing αSMA-positive cells with high frequency was formed.

FIG. 8 shows a hyaluronic acid staining image and a Sirius red staining image of a xenograft formed by transplanting immunodeficient mice (NOD/Scid mice) with a human pancreatic cancer cell line-derived pancreatic cancer organoid prepared under each condition. The Sirius red staining group was used in the detection of fibrous collagens (mainly collagens I and III) by a Sirius red staining-polarizing microscope analysis method (ellipsometry). The Sirius red staining-polarizing microscope analysis method (ellipsometry) is a visualization method that exploits the difference in collagen birefringence depending on fiber diameter, packing, and the degree of sequence, and is useful in detecting the structural change of collagens. The expression of an extracellular matrix tenascin-C was evaluated. Results of quantifying a region with high luminance are shown in the graphs of the lower panels. A hyaluronic acid stain and a strong stain of Sirius red and tenascin-C are observed within a primary lesion and a xenograft formed after transplantation of a stroma-rich cancer organoid. Collagen fiber formation is found only slightly in the inside of a xenograft formed by the dispersed transplantation (suspension) of pancreatic cancer cells or formed from a pancreatic cancer aggregate prepared from only pancreatic cancer cells, whereas a collagen-positive area is expanded in a hMSC-rich (High Stroma) pancreatic cancer organoid-derived xenograft and is closer to the primary lesion.

FIG. 9 shows results of evaluating the in vivo drug sensitivity of established human pancreatic cancer cell line (CFPAC-1) or an organoid prepared therefrom. After transplantation of the human pancreatic cancer cells or the human pancreatic cancer cell organoid to immunodeficient mice (NOD/Scid), gemcitabine was administered thereto at 3-day intervals from the point in time when the tumor size exceeded 100 mm3. Change in tumor size was plotted on a graph. It was revealed that the tumor size is reduced under a given anti-cancer drug administration condition (e.g., 10 mg/kg). Results of using this condition to evaluate the drug sensitivity of a xenograft derived from the human pancreatic cancer organoid reconstituted from the human pancreatic cancer cell line, HUVECs, and hMSCs are shown. Difference in tumor volume as between the start of administration and the completion of administration at a gemcitabine dose concentration of 10 mg/kg is shown in the right graph. Decrease in tumor volume at the time of administration of the anti-cancer drug is suppressed in groups involving hMSCs (low hMSC group and high hMSC group). Notably, an increase in tumor volume was confirmed in the group transplanted with the stroma-rich pancreatic cancer organoid (high hMSC group).

FIG. 10 The upper panel shows two culture methods of primary culture cells isolated from cancer patients. In the conventional planar culture method, there is a problem that the characteristics of epithelial cells of primary cancer cells could not be maintained. On the other hand, the cyst culture method (a method of embedding it in an extracellular matrix such as Matrigel and cultured under a given culture condition) can maintain the characteristics of epithelium of primary cancer cells. This method is reportedly capable of culturing primary pancreatic cancer cells while maintaining the properties of pancreatic cancer cells (epithelial cells). Unfortunately, a plurality of passages are difficult to perform if an expanded culture of a human pancreatic cancer specimen is attempted on the basis of the reported information. The lower panels show primary pancreatic cancer cell cyst prepared and passaged by an optimized method. 20 or more passages are possible. In the expanded culture of primary pancreatic cancer cells, multiple passages were achieved by the following optimization: I. Cell preparation condition from pancreatic cancer tissue: the pancreatic cancer tissue is digested at 37° C. for 20 minutes in a dispersion buffer (DMEM medium supplemented with 10% FBS containing Liberase™ (F. Hoffmann-La Roche, Ltd.), a ROCK inhibitor (10 μM), and DNase) and then embedded in Growth Factor reduced Matrigel. II. Method of passaging pancreatic cancer cysts in Matrigel: Matrigel containing pancreatic cancer cyst is treated with TrypLE (manufactured by Thermo Fisher Scientific Inc.) containing a ROCK inhibitor (10 μM) for 7 minutes to effect dispersion. Then, after subsequent medium exchange, the cyst is embedded in fresh Matrigel.

FIG. 11 shows a tissue image of a primary pancreatic cancer organoid obtained by three-dimensionally coculturing in vitro human primary pancreatic cancer cells, HUVECs (GFP gene-transfected), and hMSCs (having Kusabira Orange introduced therein) (FIG. 11). The left diagrams show morphology at culture day 1. The right diagrams show morphology at culture day 10. 20-day or longer culture was possible. It can be confirmed that the network topology of HUVEC cells at culture day 15 or later differs depending on the distinctive reconstitution condition (cell mixing ratio) of each organoid. The condition of pancreatic cancer cyst preparation from pancreatic cancer cells separated from pancreatic cancer cyst, HUVECs, and hMSCs is as follows: a pancreatic cancer organoid containing pancreatic cancer cyst was treated with TrypLE (Thermo Fisher Scientific Inc.) for 7 minutes to effect dispersion. Then, HUVECs and hMSCs were added thereto to prepare a primary pancreatic cancer organoid. The medium used for the primary pancreatic cancer organoid was a 1:1 liquid mixture of a basal medium for primary pancreatic cancer cells and an EGM medium (Lonza Group Ltd.). Tissue closely resembling a primary lesion in such aspects as a ductal structure and a blood vessel-like structure constituted of CK7-positive epithelial cells, is observed in the inside of a stroma-rich primary pancreatic cancer organoid. The distinct network structure of HUVECs is confirmed within the primary pancreatic cancer organoid. In addition, it is confirmed that hMSCs are present around HUVECs as if to surround the HUVECs.

FIG. 12 shows results of imaging stroma in a primary human pancreatic cancer organoid-derived xenograft. Extracellular matrices such as hyaluronic acid, fibronectin, and tenascin are abundantly detected in the cancer organoid. On the other hand, the expression of these extracellular matrices is low in a suspension comprising only pancreatic cancer cells. Results of quantifying a staining image are shown in the lower panels. The expression of these extracellular matrices closely resembling those in a primary lesion of human pancreatic cancer is observed within the primary pancreatic cancer-derived xenograft.

FIG. 13 shows results of imaging HUVECs in the inside of a primary human pancreatic cancer organoid. The distinct network formation of HUVECs was observed in the inside of a stroma-rich organoid (High stroma) as compared with a stroma-poor cancer organoid (Low stroma). The network of HUVECs is also maintained in the stroma-rich organoid at culture day 20. Presumably, hMSCs play an important role in the network formation efficiency and maintenance of HUVECs.

FIG. 14 shows a drug evaluation method for pancreatic cancer organoid created from pancreatic cancer cells transfected with a luciferase gene to express luciferase (LUC-pancreatic cancer cells), and stromal cells. The drug sensitivity of the cancer organoid can be evaluated by specifically evaluating specifically a cancer cell number within the cancer organoid on the basis of luciferase activity.

FIG. 15 shows the correlation between luciferase activity and cell number in luciferase-introduced cells (LUC-pancreatic cancer cells). The left diagram shows the fluorescence intensity of a cancer organoid at each cell number under plane culture. The right diagram shows the fluorescence intensity of a cancer aggregate created by three-dimensionally culturing pancreatic cancer cells of each cell number. In both cases, the luminescence intensity is proportional to the cancer cell number. In each, the ordinate depicts luminescence intensity (CPS), and the abscissa depicts an inoculated cell number.

FIG. 16 shows results of quantifying luciferase activity of a pancreatic cancer organoid from luciferase gene-transfected pancreatic cancer cells (LUC-pancreatic cancer cells), HUVECs, and hMSCs. The left diagrams show a GFP fluorescent image of pancreatic cancer cells in each pancreatic cancer organoid. The luminescence intensity of the pancreatic cancer organoid is shown on the right. The LUC activity was evaluated in the human pancreatic cancer organoid prepared from pancreatic cancer cells constitutively expressing luciferase, HUVECs, and hMSCs. A pancreatic cancer cell number was constant at the time of preparation of each organoid. Constant luminescence was detected regardless of the mixing ratio of HUVECs and hMSCs.

FIG. 17 shows the in vitro drug sensitivity evaluation of a pancreatic cancer organoid prepared three-dimensionally using a luciferase gene-transfected human pancreatic cancer cell line, HUVECs, and hMSCs, and plane-cultured pancreatic cancer cells (plane-alone group). In each graph, the ordinate depicts the amount of luciferase activity of the pancreatic cancer cells, and the abscissa depicts an anti-cancer drug (gemcitabine, nab-paclitaxel, or 5-FU) concentration in a medium. Two-dimensionally cultured pancreatic cancer cell exhibits high sensitivity for gemcitabine, nab-paclitaxel, and 5-FU. On the other hand, the pancreatic cancer organoid exhibit lower drug sensitivity for gemcitabine, nab-paclitaxel, and 5-FU. Among the pancreatic cancer organoids, cancer organoids with rich-stroma show lower sensitivity to anticancer drugs.

FIG. 18 shows the size of a pancreatic cancer organoid cultured in the presence of an anti-cancer drug. The upper panels show a microscope image of the pancreatic cancer organoid cultured in the presence of an anti-cancer drug, and the lower panels show results of measuring the largest projected area of each individual organoid. Decrease in aggregate size dependent on the concentration of the anti-cancer drug is found in a cancer aggregate constituted by only cancer cells. On the other hand, change in size by the concentration of the anti-cancer drug is small in the cancer organoid.

FIG. 19 shows results of analyzing the properties of residual cells in a cancer organoid cultured in the presence of an anti-cancer drug. Residual cancer cells expressing GFP and mesenchymal cells expressing αSMA are observed even after anti-cancer drug (gemcitabine) administration. Increase in positive ratio of a cancer stem cell marker Sox9 was confirmed in the residual cancer cells.

FIG. 20 Most of pancreatic cancer patients have recurrence and/or distant metastasis and exhibit poor prognosis. Whether a xenograft reconstituted from a cancer organoid could reproduce the recurrence of pancreatic cancer was studied. The upper panel shows the method. The left graph of the lower panels shows changes in tumor size in a gemcitabine administration period and discontinuation period. The right diagrams show a macro photograph of the xenograft in each period. A xenograft formed after cancer organoid transplantation exhibits decrease in tumor size after treatment with a high concentration of gemcitabine, but experiences an increase in tumor size upon discontinuation of the gemcitabine administration. By contrast, a cancer suspension-derived xenograft exhibits a less variable tumor size after the treatment.

FIG. 21 shows a tissue image of residual cancer tissue after administration of an anti-cancer drug for 30 days to a xenograft formed after transplantation of a pancreatic cancer organoid reconstituted using an established pancreatic cancer cell line (CFPAC-1) and stromal cells. The dose of gemcitabine is set to 10 mg/kg. Decrease in tumor size is found in the GEM administration group (e.g., 30 mg/kg), whereas it is confirmed that the incidence of cancer cells (CK7-positive cells) within the tissue is increased. Ki67-positive cells are present with high incidence within the residual pancreatic cancer tissue after the anti-cancer drug administration. A xenograft reconstituted from a stroma-rich pancreatic cancer organoid exhibits high resistance to an anti-cancer drug.

FIG. 22 shows results of administering an anti-cancer drug (e.g., 50 mg/kg) for 30 days to a xenograft formed after transplantation of a pancreatic cancer organoid reconstituted using an established pancreatic cancer cell line (CAPAN-2) and stromal cells, and immunostaining residual cancer tissue. The xenograft formed after cancer organoid transplantation has high incidence of Ki67-positive cells and low incidence of Caspase-3-positive cells, as compared with a cancer suspension transplantation group. Also, the frequency of cells expressing cancer stem cell marker Sox9 is high. Xenografts formed after pancreatic cancer organoid transplantation are useful as drug discovery system for Sox9-positive pancreatic cancer stem cells.

FIG. 23 shows the expression of a drug transporter in residual cancer tissue after administration of an anti-cancer drug (e.g., 10 mg/kg) for 30 days to a xenograft formed after transplantation of a pancreatic cancer organoid reconstituted using an established pancreatic cancer cell line (CFPAC-1) and stromal cells. This figure shows results of analyzing the expression of an ABC transporter (e.g., ABCG2) reportedly involved in anti-cancer drug resistance. Pancreatic cancer cells that express ABCG2 and exhibit cancer stem cell-like phenotypes remain with high frequency in the pancreatic cancer organoid transplantation group after the anti-cancer drug administration. On the other hand, the incidence of ABCG2-positive cells is low in a cancer aggregate transplantation group.

FIG. 24 shows an imaging photograph of a vascular network within a xenograft formed after cancer organoid transplantation. This figure shows a tissue image after transplantation of a cancer organoid into a cranial window prepared in the mouse head. A group transplanted with an organoid constituted of KO-HUVECs and hMSCs was set as a control for transplantation. In order to visualize the vascular network within the cranial window, high-molecular-weight fluorescent dextran (M.W. 2,000 kDa) was injected from the mouse tail vein, and images were captured within 15 minutes. The upper panels of the right diagrams show an image of cancer cells expressing the fluorescent gene and blood vessels labeled with high-molecular-weight fluorescent dextran. A tumor vascular structure that exhibits heterogeneous and excessive branches is confirmed within a xenograft formed after pancreatic cancer organoid transplantation. The extravasation of low-molecular dextran is further detected in the xenograft after pancreatic cancer organoid transplantation.

FIG. 25 Vascular leakiness within a xenograft formed after transplantation of a cancer organoid into a cranial window was evaluated. This figure shows results of Evans blue staining. Blood vessel leakage is increased in a xenograft formed after stroma-rich cancer organoid transplantation.

FIG. 26 shows a tissue image of a xenograft formed after primary pancreatic cancer organoid transplantation. After primary cancer organoid transplantation, pancreatic cancer tissue having a tubular structure and abundant stroma is reconstituted. The xenograft formed after primary pancreatic cancer organoid transplantation exhibits a tissue image characteristic of human pancreatic ductal adenocarcinoma, while αSMA-positive mesenchymal cells are abundantly present in areas around the ductal structure. The lower panels show results of quantification.

FIG. 27 Primary cancer organoids were respectively reconstituted from two primary cancer cell lines prepared from different pancreatic cancer patients, and evaluated for their drug sensitivity in vitro. Pancreatic cancer cells transfected with luciferase gene were used for experiments. The primary cancer organoids exhibit high drug resistance as compared with an aggregate of only primary cancer cells (cancer cyst group).

FIG. 28 A primary cancer organoid was prepared from a primary cancer cell line, and a xenograft was reconstituted in immunodeficient mice in vivo. Gemcitabine administration was performed targeting this xenograft, and variation in tumor size was observed. Changes in tumor size are shown graphically. The primary cancer organoid transplantation group exhibits high treatment resistance as compared with a group transplanted with only primary cancer cells (cancer cyst transplantation group).

FIG. 29 A lung cancer organoid prepared three-dimensionally using a human lung cancer cell line (A549 cells) expressing luciferase gene and EGFP, as well as HUVECs and hMSCs, and a three-dimensional aggregate composed of only pancreatic cancer cells were evaluated for their in vitro drug sensitivity. The left diagrams show a fluorescence phase-contrast microscope image of the lung cancer organoid. In the graph of the right diagram, the ordinate depicts the amount of luciferase activity of the lung cancer cells, and the abscissa depicts an anti-cancer drug (gemcitabine) concentration in the medium. The lung cancer cell aggregate exhibits high sensitivity for gemcitabine. On the other hand, the pancreatic cancer organoid culture groups have lower drug sensitivity for gemcitabine. Among the pancreatic cancer organoid groups, the group involving hMSCs and HUVECs with high incidence (High stroma group) has even lower sensitivity for the anti-cancer drug. In lung cancer as well, a stroma-rich cancer organoid exhibits drug resistance.

FIG. 30 A primary pancreatic cancer organoid or a primary pancreatic cancer suspension was transplanted to immunodeficient mice, and after confirmation of tumor formation, the mice were exposed to radiation (carbon ion beam). This figure shows changes in tumor volume after the irradiation. A xenograft formed from the cancer organoid is confirmed to exhibit resistance to carbon ion beam irradiation.

FIG. 31 shows how the drug sensitivity of a primary human pancreatic cancer organoid correlates with patient prognosis. The drug sensitivity of a pancreatic cancer organoid is related to postoperative recurrence.

### MODE FOR CARRYING OUT THE INVENTION

Hereinafter, embodiments of the present invention will be described in more detail.

The present invention provides a reconstituted cancer organoid reproducing a cancer microenvironment.

In the present invention, the “cancer organoid” is a cell aggregate constituted from cancer cells and other cells. The cancer organoid is capable of reproducing the intercellular interaction between a plurality of cells. The cancer organoid of the present invention reproduces a cancer microenvironment and is rich in stroma, for example.

There are several approaches for quantifying the richness of stroma in the cancer organoid.

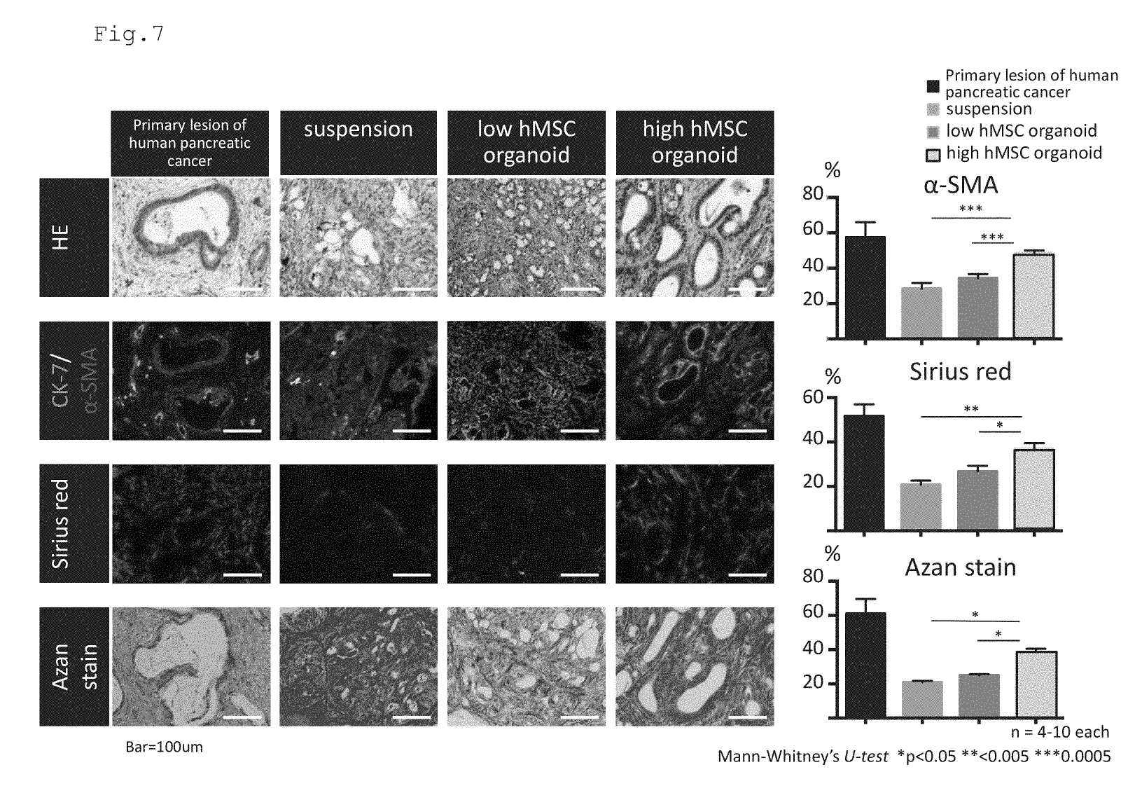

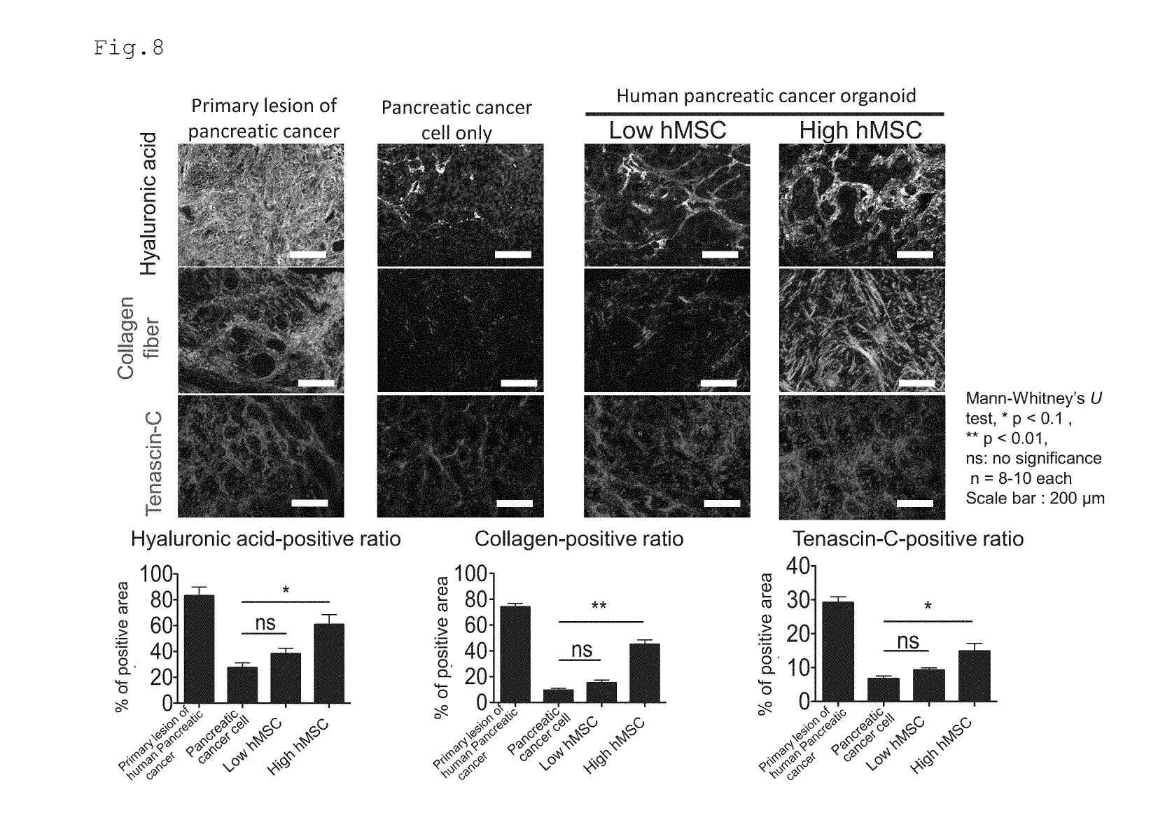

a: Quantification by immunostaining using a mesenchymal cell marker (αSMA) as an index (see FIG. 7). The positive ratio was approximately 59% for a pancreatic cancer organoid (10:7:20) prepared in Examples mentioned later, as compared with a primary lesion (approximately 61%). The cancer organoid of the present invention may have an immunostain-positive ratio of 1 to 1000%, preferably 10 to 500%, more preferably 10 to 300%, in this quantification method.
b: Quantification of extracellular matrices (e.g. hyaluronic acid, collagens, etc. in the case of pancreatic cancer) within stroma (the collagens can be qualitatively analyzed by Sirius red staining and quantitatively analyzed by polarizing microscope image analysis after the Sirius red staining (see FIG. 8). In Examples mentioned later, the positive ratio was approximately 44% for a pancreatic cancer organoid (10:7:20), as compared with a primary lesion (approximately 74%). The cancer organoid of the present invention, as assayed by this quantification method, may have a positive ratio of 1 to 1000%, preferably 10 to 500%, more preferably 10 to 300%.
c: The hardness of tissue is increased as hyaluronic acid or collagens accumulate within stroma. Thus, the richness of stroma may be determined with tissue hardness taken as an index.

In many cases, cancer tissue has a portion called stroma, in addition to cancer cells. In the stroma, mesenchymal cells including fibroblasts, as well as many types of cells including cells constituting blood vessels, lymph ducts, nerves, and the like (blood cells, vascular cells, immunocytes, etc.), and cells responsible for inflammation (inflammatory cells), and connective tissue composed of collagens and the like between these cells are present to form a characteristic structure. This structure is called cancer microenvironment.

The cancer organoid of the present invention may reproduce a cancer microenvironment comprising cancer stroma. The cancer organoid of the present invention may further reproduce a ductal structure, in addition to the cancer microenvironment. The ductal structure may be formed by cancer cells having epithelial properties.

The present invention also provides a reconstituted cancer organoid reproducing at least one selected from the group consisting of treatment resistance, invasion or metastasis, and recurrence of cancer. Examples of the treatment resistance of cancer can include drug sensitivity, radiation sensitivity, immunotherapy sensitivity, nutrition therapy sensitivity and the like. The “recurrence of cancer” means re-appearance of cancer after resection, re-appearance of cancer that has disappeared as a result of anti-cancer drug treatment, radiation treatment, immunotherapy, nutrition therapy, or a combination thereof appears again, or re-enlargement of tumor that has decreased in size, and conceptually includes not only the occurrence of cancer at or near the treated site but discovery as metastasis at a different site.

The present invention further provides a reconstituted cancer organoid allowing prognostic prediction of cancer.

The type of the cancer is not particularly limited and may be any cancer such as liver cancer, kidney cancer, malignant brain tumor, pancreatic cancer, stomach cancer, lung cancer or the like. In Examples mentioned later, a pancreatic cancer organoid was prepared.

The cancer organoid of the present invention can be prepared by coculturing cancer cells with mesenchymal cells and vascular endothelial cells. The culture may be three-dimensional (3D) culture. 3D culture techniques suitable for the reconstitution of the cancer organoid of the present invention have been reported in, for example, Nature, 25; 499 (7459): 481-4, 2013, Nat Protoc. 9 (2): 396-409, 2014, and Cell Stem Cell, 7; 16 (5): 556-65, 2015.

The cancer cells may be a pre-existing cancer cell line or may be a primary cancer cell line established using a cancer tissue separated from a primary lesion of human cancer. The type of the cancer is not particularly limited and may be any cancer such as liver cancer, kidney cancer, malignant brain tumor, pancreatic cancer, stomach cancer, lung cancer or the like. Although human-derived cancer is typically used, cancer cells derived from nonhuman animals (e.g., animal for use as a laboratory animal, a pet animal, a working animal, a racehorse, a fighting dog, or the like, specifically, mouse, rat, rabbit, pig, dog, monkey, cattle, horse, sheep, chicken, shark, ray, elephant fish, salmon, shrimp, crab, etc.)—may also be used.

In the present invention, the “vascular endothelial cells” refers to cells that constitute vascular endothelium, or cells that can differentiate into such cells. Whether certain cells are vascular endothelial cells can be confirmed by examining whether they express a marker protein, for example, TIE2, VEGFR-1, VEGFR-2, VEGFR-3, and/or CD41 (cells expressing any one or two or more of these marker proteins can be judged as being vascular endothelial cells). The vascular endothelial cells to be used in the present invention may be differentiated or undifferentiated. Whether the vascular endothelial cells are differentiated cells or not can be confirmed using CD31 and CD144. Among the terms employed by those skilled in the art, endothelial cells, umbilical vein endothelial cells, endothelial progenitor cells, endothelial precursor cells, vasculogenic progenitors, hemangioblasts (H J. Joo, et al., Blood. 25; 118 (8): 2094-104. (2011)), and the like are included in the vascular endothelial cells according to the present invention. The vascular endothelial cells are preferably umbilical vein-derived vascular endothelial cells. The vascular endothelial cells can be collected from blood vessels or can be prepared according to a method known in the art from pluripotent stem cells such as induced pluripotent stem cells (iPS cells), embryonic stem cells (ES cells) or the like. Although human-derived vascular endothelial cells are typically used, vascular endothelial cells derived from nonhuman animals (e.g., animal for use as a laboratory animal, a pet animal, a working animal, a racehorse, a fighting dog, or the like, specifically, mouse, rat, rabbit, pig, dog, monkey, cattle, horse, sheep, chicken, shark, ray, elephant fish, salmon, shrimp, crab, etc.) may also be used.

In the present invention, the “mesenchymal cells” are typically connective tissue cells that reside in mesoderm-derived connective tissue and form a support structure of cells functioning in tissue, and conceptually also include cells that are destined, but are yet, to differentiate into mesenchymal cells. The mesenchymal cells to be used in the present invention may be differentiated or undifferentiated. Whether certain cells are undifferentiated mesenchymal cells or not can be confirmed by examining whether they expressa marker protein, for example, Stro-1, CD29, CD44, CD73, CD90, CD105, CD133, CD271, and/or Nestin (cells expressing any one or two or more of these marker proteins can be judged as being undifferentiated mesenchymal cells). In addition, mesenchymal cells expressing none of these markers can be judged as being differentiated mesenchymal cells. Among the terms employed by those skilled in the art, mesenchymal stem cells, mesenchymal progenitor cells, mesenchymal cells (R. Peters, et al., PLoS One. 30; 5 (12): e15689. (2010)), and the like are included in the mesenchymal cells according to the present invention. The mesenchymal cells are preferably bone marrow-derived mesenchymal cells (particularly, mesenchymal stem cells). The mesenchymal cells can be collected from bone marrow, fat tissue, placenta tissue, umbilical cord tissue, tissue of dental pulp, or the like, or can be prepared according to a method known in the art from pluripotent stem cells such as induced pluripotent stem cells (iPS cells) or embryonic stem cells (ES cells). Although human-derived mesenchymal cells are typically used, undifferentiated mesenchymal cells derived from nonhuman animals (e.g., animal for use as a laboratory animal, a pet animal, a working animal, a racehorse, a fighting dog, or the like, specifically, mouse, rat, rabbit, pig, dog, monkey, cattle, horse, sheep, chicken, shark, ray, elephant fish, salmon, shrimp, crab, etc.) may be used.

The culture ratio among the three types of cells in the coculture is not particularly limited as long as it falls within a range that permits cancer organoid formation. The cell number ratio is preferably cancer cells/vascular endothelial cells/mesenchymal cells=10:1 to 100:1 to 100, more preferably cancer cells/vascular endothelial cells/mesenchymal cells=10:1 to 100:5 to 100. Approximately 200,000 cancer cells, approximately 140,000 vascular endothelial cells, and approximately 200,000 mesenchymal cells can be cocultured to form a cancer organoid having a size on the order of 50 to 50000 μm.

The medium for use in the culture may be any medium as long as the cancer organoid can be formed. For example, a medium for vascular endothelial cell culture, a medium for cancer cell culture, or a mixture of these two media is preferably used. Any medium for vascular endothelial cell culture may be used, and a medium containing at least one of hEGF (recombinant human epithelial cell growth factor), VEGF (vascular endothelial cell growth factor), hydrocortisone, bFGF, ascorbic acid, IGF1, FBS, antibiotics (e.g., gentamicin and amphotericin B), heparin, L-glutamine, phenol red, and BBE is preferably used. As the medium for vascular endothelial cell culture, EGM-2 BulletKit (manufactured by Lonza Group Ltd.), EGM BulletKit (manufactured by Lonza Group Ltd.), VascuLife EnGS Comp Kit (manufactured by Lifeline Cell Technology (LCT)), Human Endothelial-SFM Medium (manufactured by Thermo Fisher Scientific Inc.), or Human Microvascular Endothelial Cell Growth Medium (manufactured by Toyobo Co., Ltd.) can be used. Any medium for cancer cell culture may be used, and examples thereof include DMEM medium. It has been confirmed that a medium of EGM:DMEM=1:1 is suitable for the preparation of a pancreatic cancer organoid (see Examples mentioned later).

For the culture of the cells, it is not necessary to use a scaffold material. The mixture of the three types of cells may be cultured on a gel-like support that permits contraction of the mesenchymal cells.

The contraction of the mesenchymal cells can be confirmed by three-dimensional tissue formation that is morphologically observed (either under microscope or by the naked eye), or by showing that the cells have a sufficient strength to keep the shape of tissue during recovery with a medicine spoon or the like (Takebe et al. Nature 499 (7459), 481-484, 2013)).

The support may be a gel-like substrate having appropriate hardness (e.g., Young's modulus: 200 kPa or lower (in the case of, for example, a Matrigel-coated gel having a flat shape), though the appropriate hardness of the support may vary depending on the coating and shape). Examples of such a substrate can include, but are not limited to, hydrogels (e.g., acrylamide gel, gelatin, and Matrigel). The hardness of the support is not necessarily required to be uniform, and a spatial or temporal gradient may be set in the hardness, or the support may be patterned, according to the shape, size, and amount of the assembly of interest. In the case where the hardness of the support is uniform, the hardness of the support is preferably 100 kPa or lower, more preferably 1 to 50 kPa. The gel-like support may have flat surfaces, or the culture side of the gel-like support may have a U- or V-shaped cross section. If the culture side of the gel-like support has a U- or V-shaped cross section, cells gather on the culture surface of the support so that a cell assembly is advantageously formed by a smaller number of cells and/or tissues. Alternatively, the support may be modified chemically or physically. Examples of the modifying material can include Matrigel, laminin, entactin, collagen, fibronectin, vitronectin and the like.

One example of the case where a spatial gradient is set in the hardness of the gel-like culture support is a gel-like culture support that is harder in the central part than in the peripheral part. The appropriate hardness of the central part is 200 kPa or lower, and the hardness of the peripheral part may be such that it is softer than the central part. The appropriate hardness of the central part and the peripheral part of the support may vary depending on the coating and shape. Another example of the case where a spatial gradient is set in the hardness of the gel-like culture support is a gel-like culture support that is harder in the peripheral part than in the central part.

One example of the patterned gel-like culture support is a gel-like culture support having one or more patterns that are harder in the central part than in the peripheral part. The appropriate hardness of the central part is 200 kPa or lower, and the hardness of the peripheral part may be such that it is softer than the central part. The appropriate hardness of the central part and the peripheral part of the support may vary depending on the coating and shape. Another example of the patterned gel-like culture support is a gel-like culture support having one or more patterns that are harder in the peripheral part than in the central part. The appropriate hardness of the peripheral part is 200 kPa or lower, and the hardness of the central part may be such that it is softer than the peripheral part. The appropriate hardness of the central part and the peripheral part of the support may vary depending on the coating and shape.

The culture temperature is not particularly limited and is preferably 30 to 40° C., more preferably 37° C.

The culture period is not particularly limited and is preferably 1 to 60 days, more preferably 1 to 7 days.

The present inventors have also successfully established a primary cancer cell line using a cancer tissue separated from a primary lesion of human cancer and prepared a cancer organoid using this primary cancer cell line. Accordingly, the present invention also provides a method for preparing a cancer organoid from a primary cancer cell line. This method comprises: digesting a cancer tissue in the presence of a proteolytic enzyme and a Rho kinase (ROCK) inhibitor and then obtaining an aggregate of cancer cells; passaging the aggregate and then separating the cancer cells; and coculturing the cancer cells with mesenchymal cells and vascular endothelial cells to form the cancer organoid. In the method of the present invention, the cancer tissue may be digested in the presence of deoxyribonuclease together with the proteolytic enzyme and the Rho kinase inhibitor. The cancer organoid may reproduce a cancer microenvironment. The cancer microenvironment may comprise cancer stroma. The cancer organoid may further reproduce a ductal structure. The ductal structure may be formed by cancer cells having epithelial properties. The cancer organoid may reproduce at least one selected from the group consisting of treatment resistance, invasion or metastasis, and recurrence of cancer. Examples of the treatment resistance of cancer can include drug sensitivity, radiation sensitivity, immunotherapy sensitivity, nutrition therapy sensitivity and the like. The type of the cancer is not particularly limited and may be any cancer such as liver cancer, kidney cancer, malignant brain tumor, pancreatic cancer, stomach cancer, lung cancer or the like. In Examples mentioned later, a pancreatic cancer organoid and a lung cancer organoid were prepared. A strong aggregation of pancreatic cancer organoid with a cell mixing ratio having a high proportion of mesenchymal cells was observed (see Examples mentioned later).

For the digestion of the cancer tissue, the cancer tissue may be incubated at 37° C. for an appropriate time (20 minutes in Examples mentioned later) in a medium (e.g., DMEM medium) supplemented with the proteolytic enzyme and the Rho kinase inhibitor (the medium may be further supplemented with deoxyribonuclease). The concentration of the Rho kinase inhibitor in the medium may be approximately 10 μM. Examples of the Rho kinase inhibitor can include Y-27632 (R&D Systems, Inc.) (in Examples mentioned later, Y-27632 (R&D Systems, Inc.) was used). The medium may be supplemented with FBS.

The aggregate of cancer cells (cancer cyst) may be passaged in such a state that it is embedded in a gel (e.g., Matrigel). A dispersing solution (e.g., TrypLE (Thermo Fisher Scientific Inc.)) supplemented with the Rho kinase inhibitor may be used for dispersion of the cancer cyst at the time of passage. After subsequent medium replacement, the dispersed cancer cyst may be embedded in a fresh gel.

The cancer cyst thus passaged may be treated with a dispersing solution (e.g., TrypLE (Thermo Fisher Scientific Inc.)) and then cocultured with vascular endothelial cells and mesenchymal cells. The coculture of the cancer cells with vascular endothelial cells and mesenchymal cells is as mentioned above.

A xenograft reproducing a cancer microenvironment can be prepared by transplanting a nonhuman animal with a reconstituted cancer organoid reproducing a cancer microenvironment. Accordingly, the present invention also provides a method for preparing a xenograft reproducing a cancer microenvironment, comprising transplanting a nonhuman animal with a reconstituted cancer organoid reproducing a cancer microenvironment . The present invention also provides a xenograft reproducing a cancer microenvironment, the xenograft being obtained by transplanting a nonhuman animal with a reconstituted cancer organoid reproducing a cancer microenvironment. The cancer microenvironment may comprise cancer stroma. The reconstituted cancer organoid may further reproduce a ductal structure. Alternatively, the xenograft itself may further reproduce a ductal structure. The ductal structure may be formed by cancer cells having epithelial properties. The xenograft may reproduce at least one selected from the group consisting of treatment resistance, invasion or metastasis, and recurrence of cancer. The cancer organoid may be a cancer organoid reconstituted from primary cancer cells or may be a cancer organoid reconstituted from a pre-existing cancer cell line. The present invention also provides a cancer organoid-derived xenograft reproducing at least one selected from the group consisting of treatment resistance, invasion or metastasis, and recurrence of cancer. The present invention also provides a cancer organoid-derived xenograft reproducing expression of a drug transporter. The present invention further provides a cancer organoid-derived xenograft having tumor vessels. The present invention also provides a cancer organoid-derived xenograft reproducing drug leakage characteristic of tumor vessels. These cancer organoid-derived xenografts can each be prepared by transplanting a nonhuman animal with a cancer organoid formed by the coculture of cancer cells with mesenchymal cells and vascular endothelial cells. The type of the cancer is not particularly limited and may be any cancer such as liver cancer, kidney cancer, malignant brain tumor, pancreatic cancer, stomach cancer, lung cancer or the like. In Examples mentioned later, a xenograft was prepared from a pancreatic cancer organoid. A xenograft prepared from a pancreatic cancer organoid with a cell mixing ratio having a high proportion of mesenchymal cells was rich in stroma and tended to exhibit lower drug sensitivity (see Examples mentioned later). Examples of the nonhuman animal as a recipient for the transplantation can include, but are not limited to, mice, rats, rabbits, pigs, dogs, monkeys, cattle, horses, sheep, and chickens.

The cancer organoid and the xenograft of the present invention can be used in the evaluation of at least one selected from the group consisting of treatment resistance, invasion or metastasis, and recurrence of cancer. Accordingly, the present invention also provides a method for evaluating treatment resistance of cancer using the cancer organoid and/or the xenograft. The present invention also provides a method for evaluating invasion or metastasis using the cancer organoid and/or the xenograft. The present invention further provides a method for evaluating recurrence using the cancer organoid and/or the xenograft.

In the case of evaluating the treatment resistance of cancer using the cancer organoid, the cancer organoid may be subjected to a procedure equivalent to the treatment of cancer (e.g., addition of a drug, exposure to radiation, addition of an immunotherapeutic, or addition of a nutrient). After a lapse of an appropriate time, the number of surviving cancer cells is counted, and an IC50 value may be calculated.

In the case of evaluating the treatment resistance of cancer using the xenograft, the cancer organoid is transplanted to a nonhuman animal. At the point in time when the volume of the formed xenograft becomes an appropriate size, the treatment of cancer is started. After administration with appropriate frequency, the xenograft is resected, and its volume may be measured.

Examples of the cancer therapeutic include pre-existing cancer therapeutics (also including radiation) and candidate compounds for cancer therapeutics.

In the case of evaluating the invasion or metastasis of cancer using the cancer organoid, cell migration from the cancer organoid may be observed by use of migration and invasion assay using, for example, Transwell. In the case of evaluating the invasion or metastasis of cancer using the xenograft, the cancer organoid is transplanted to a nonhuman animal. After a lapse of an appropriate time after the volume of the formed xenograft becomes an appropriate size, a cancer cell colony or cancer cells may be observed within tissue predicted to have distant metastasis.

In the case of evaluating the recurrence of cancer using the cancer organoid, the cancer organoid may be subjected to a procedure equivalent to the treatment of cancer (e.g., addition of a drug, exposure to radiation, addition of an immunotherapeutic, or addition of a nutrient). After observation of disappearance of cancer cells or decrease in cancer cell number, the procedure equivalent to the treatment of cancer is discontinued. After a lapse of an appropriate time, the number of surviving cancer cells or the size of the cancer organoid may be counted.

In the case of evaluating the recurrence of cancer using the xenograft, the cancer organoid is transplanted to a nonhuman animal. At the point in time when the volume of the formed xenograft becomes an appropriate size, the treatment of cancer is started. After observation of disappearance of the xenograft or decrease in xenograft volume by administration with appropriate frequency, the treatment of cancer is discontinued. After a lapse of an appropriate time, the volume of the xenograft or the number of constituent cells may be measured.

The method for evaluating invasion or metastasis of cancer and the method for evaluating recurrence of cancer according to the present invention can also be used in the screening for a cancer therapeutic. This screening can lead to the discovery of a drug for the treatment and/or prevention of invasion or metastasis of cancer or a drug effective for the prevention of recurrence of cancer.

It has been shown that the drug sensitivity of a primary cancer organoid is related to postoperative recurrence in a patient (Examples mentioned later). This suggests that the treatment resistance of a cancer organoid and a xenograft prepared from the cancer organoid correlates with patient prognosis. Accordingly, the present invention also provides a method for conducting prognostic prediction of cancer using the cancer organoid and/or the xenograft. In the case where a cancer organoid and/or a xenograft derived from cancer cells of a patient is treatment-sensitive, the patient is predicted to be free from postoperative recurrence. In the case where a cancer organoid and/or a xenograft derived from cancer cells of a patient is treatment-resistant, the patient is predicted to suffer postoperative recurrence.

The present invention also provides a nonhuman animal carrying the xenograft. The xenograft has been mentioned above. Examples of the nonhuman animal can include, but are not limited to, mice, rats, rabbits, pigs, dogs, monkeys, cattle, horses, sheep, and chickens. The nonhuman animal of the present invention can be used in the evaluation of treatment resistance, invasion or metastasis, or recurrence of cancer, prognostic prediction of cancer, etc.

### EXAMPLES

Hereinafter, the present invention will be specifically described with reference to Examples. However, the present invention is not intended to be limited to these Examples.

### Example 1

### 1. Material and Method

### 1-1. Human Cell

The pre-existing human pancreatic cancer cell lines used were CFPAC-1 (ATCC: CRL-1918), PANC-1 (provided by RIKEN BRC: RCB2095), and SW1990 (ATCC: CRL-2172). CFPAC-1 is a cell line established from a lesion with liver metastasis of a 26-year old male. PANC-1 is a cell line established from a primary lesion of a patient of unknown age and sex. SW1990 is a cell line established from a lesion with spleen metastasis of a 56-year old male. In the present study, these cell lines were each introduced and then used at a passage number of 10 or less in experiments.

Also, human umbilical vein endothelial cells (HUVECs), human mesenchymal stem cells (hMSCs), and these cells transfected with fluorescent reporter gene (EGFP or Kusabira Orange) or luciferase gene were used.

### 1-2. In Vitro Drug Sensitivity Evaluation of Pre-Existing Human Pancreatic Cancer Cell Line

Each pre-existing human pancreatic cancer cell line was inoculated at 5×10³ cells/well on a 96-well plate, and gemcitabine (10⁻¹² to 10⁻³ M) was added thereto 24 hours later. Nuclear staining was performed 72 hours after the gemcitabine addition. A cell number was measured using IN Cell Analyzer 2000, and an IC50 value was calculated. In order to specifically detect cancer cells within an organoid and calculate a cancer cell number, luciferase gene-transfected cancer cells were established and used in analysis. A cancer organoid was formed from the luciferase gene-transfected cancer cells, and luminescence was measured in the presence of a luminescent substrate (e.g., Luciferase Assay System from Promega Corp.) to evaluate the number of cancer cells present.

### 1-3. In Vivo Drug Sensitivity Evaluation of Pre-Existing Human Pancreatic Cancer Cell Line

Each pre-existing human pancreatic cancer cell line was subcutaneously transplanted at 1×10⁶ cells to each of 4- to 10-week old female immunodeficient mice (NOD/Scid mice) to prepare a xenograft. The number of xenografts formed and the volume of each xenograft were measured over time. The volume was calculated according to (minor axis×minor axis×major axis/2) mm³. The intraperitoneal administration of gemcitabine was started from the point in time when the volume of the xenograft formed exceeded 100 mm³. The dose concentration of gemcitabine was set to 100 mg/kg, 0 mg/kg, 5 mg/kg, or 10 mg/kg, and gemcitabine was administered once every three days for 3 weeks. Then, the xenograft was resected.

### 1-4. Provided Clinical Specimen of Human Pancreatic Cancer

The clinical specimens of human pancreatic cancer (CRT-treated specimens and non-CRT-treated specimens) were obtained with the approval of the ethical review committee of Yokohama City University. The clinical specimens were collected from patients who gave preoperative informed consent to doctors in charge.

### 1-5. Preparation of Human Pancreatic Cancer Cell Line Organoid

A 1:1 mixed solution of DMEM and EGM containing 10% FBS was mixed with Matrigel, and the mixture was added to each well of a 48-well plate and incubated at 37° C. for 30 minutes. A mixed cell suspension of a human pancreatic cancer cell line, human umbilical vein endothelial cells (HUVECs), and human mesenchymal stem cells (hMSCs) was added thereto, followed by incubation at 37° C. for 5 minutes. The cells were mixed at a cancer/HUVEC/hMSC ratio (C:H:M ratio) of 10:0:0, 10:7:1, 10:7:20, 10:7:0, or 10:0:20 with the cell number of the pre-existing human pancreatic cancer cell line set to 2×10⁵ cells. Then, a 1:1 mixed solution of EGM and DMEM was added to each well, followed by incubation at 37° C.

On the other hand, for the large-scale preparation of pancreatic cancer cell organoids having a uniform size, human pancreatic cancer cells, HUVECs, and hMSCs were cocultured using a three-dimensional culture vessel (e.g., ELPLASIA plate from Kuraray Co., Ltd.) to reconstitute a human pancreatic cancer cell line organoid. The pancreatic cancer cells of each line were inoculated at 1×10⁴ cells on each well of a 96-well plate which was also inoculated with arbitrary numbers of HUVECs and hMSCs to reconstitute a cancer organoid. The mixing ratio of the cancer cells, HUVECs, and hMSCs was set to 10:0:0, 10:7:1, 10:7:20, 10:7:0, or 10:0:20.

### 1-6. Time Lapse Analysis of Human Pancreatic Cancer Cell Organoid

The process of formation of a pancreatic cancer organoid was observed for 72 hours from the start of culture using a stereoscopic microscope having time lapse photography functions while the culture plate was warmed at 37° C. In order to observe the process of formation of a pancreatic cancer organoid at a cellular level, imaging was performed using a confocal microscope. A cancer organoid was reconstituted using GFP gene-transfected HUVECs, Kusabira Orange gene-transfected hMSCs, and cancer cells of each line, and green fluorescent and red fluorescent images were captured.

### 1-7. Evaluation of Ability to Form Tumor

A prepared pre-existing human pancreatic cancer cell line organoid was subcutaneously transplanted after 24-hour culture to each of 4- to 10-week old female NOD/Scid mice to prepare a xenograft. The number of xenografts formed and the volume of each xenograft were measured over time. The volume was calculated according to (minor axis×minor axis×major axis/2) mm³.

### 1-8. Drug Sensitivity Evaluation of Human Pancreatic Cancer Organoid-Derived Xenograft

A xenograft was prepared by the subcutaneous transplantation of a human pancreatic cancer cell organoid. Then, the intraperitoneal administration of gemcitabine was started from the point in time when the volume of the xenograft exceeded 100 mm³. The dose concentration of gemcitabine was set to 0 mg/kg, 5 mg/kg, or 10 mg/kg, and the administration frequency and period were set to once every three days for 3 weeks. The volume of the xenograft was measured at appropriate times. Also, tissue was resected at appropriate times and histologically evaluated.

### 1-9. Paraffin Section Preparation

A xenograft was resected, washed with phosphate buffered saline (PBS), and then fixed overnight at 4° C. using 4% paraformaldehyde (PFA). The fixed tissue was washed with PBS for 10 minutes three times, followed by replacement treatment with ethanol and xylene in an automatic embedding apparatus. Then, the tissue was embedded in paraffin to prepare a paraffin block. The prepared paraffin block was sliced into a thickness of 4 to 6 using a microtome, and the slice was placed on a glass slide (Matsunami Glass Ind., Ltd.) and stretched and dried in a paraffin stretching plate.

### 1-10. HE (Haematoxylin-Eosin) Staining

A thin paraffin section was incubated at 72° C. for 20 minutes and then deparaffinized with xylene for 5 minutes three times. Next, the section was hydrophilized with a descending ethanol series (100 to 50%). After replacement with MilliQ, nuclear staining was performed with haematoxylin (Wako Pure Chemical Industries, Ltd.) for 10 minutes. After confirmation of sufficient staining, the tissue section was washed with running water for 10 minutes. Then, the cytoplasm was stained with eosin (Muto Pure Chemicals Co., Ltd.) for 1 minute. After confirmation of sufficient staining, the tissue section was washed with pure water. Next, the section was dehydrated with an ascending ethanol series (50 to 100%), followed by clearing treatment with xylene for 5 minutes three times. Finally, the section was mounted on a glass slide (Matsunami Glass Ind., Ltd.).

### 1-11. Immunohistochemical Staining

A paraffin section was deparaffinized, then dipped in a citrate buffer, and activated at 121° C. for 20 minutes. After washing with PBS containing 0.05% Tween 20 (PBST) for 5 minutes three times, a buffer for blocking (Dako Japan Co., Ltd.) was added to the section, and blocking reaction was performed at room temperature for 1 hour. Next, a primary antibody solution was added thereto and reacted overnight at 4° C. After the primary antibody (anti-EpCAM antibody, anti-αSMA antibody, anti-cytokeratin 7 (CK7) antibody, anti-CD31 antibody, or anti-laminin antibody) reaction, the section was washed with PBST for 5 minutes three times. A secondary antibody solution diluted with a buffer solution was added thereto and reacted at room temperature for 1 hour in the dark. After the secondary antibody reaction, the section was washed with PBST for 5 minutes three times and mounted on a glass slide using a mounting agent containing a DAPI staining solution (Wako Pure Chemical Industries, Ltd.).

### 1-12. Imaging of Immunostained Slide

An immunostained glass slide was observed using an upright fluorescence microscope (Carl Zeiss AG).

### 1-13. Sirius Red Staining

Tissue was stained using a Sirius red staining reagent (Muto Pure Chemicals Co., Ltd.). The staining method followed the manual of the staining reagent. After the staining, images were captured using an upright microscope. The tissue thus stained with Sirius red was further analyzed using a polarizing microscope (Olympus Corp.), and images were captured.

### 1-14. Separation and Culture of Primary Pancreatic Cancer Cell

Pancreatic cancer tissue was digested at 37° C. for 20 minutes in a dispersion buffer (DMEM medium containing Liberase™ (F. Hoffmann-La Roche, Ltd.), a ROCK inhibitor (10 μM), and 10% FBS) and then embedded in Growth Factor reduced Matrigel. Then, culture was performed at 37° C. Pancreatic cancer cyst was passaged by the following method: Matrigel containing the pancreatic cancer cyst was treated with TrypLE (Thermo Fisher Scientific Inc.) containing a ROCK inhibitor (10 μM) for 7 minutes to effect dispersion. After subsequent medium replacement, the dispersed cancer cyst was embedded in fresh Matrigel.

1-15. Reconstitution of Pancreatic Cancer Organoid from Primary Pancreatic Cancer Cell

Pancreatic cancer cyst was dispersed by the same approach as in the passage and then three-dimensionally cocultured with HUVECs and hMSCs using Matrigel. The three-dimensional coculture method abides by the method for a pancreatic cancer organoid from a pancreatic cancer cell line. The primary pancreatic cancer organoid was cultured by mixing a basal medium used in the previous report (Cell, 2015) and EGM at 1:1, and then embedding the mixture in Matrigel, followed by incubation at 37° C.

### Composition of Culture Solution:

AdDMEM/F12 medium
+Growth Factor reduced Matrigel
+HEPES (Thermo Fisher Scientific Inc.) (final concentration: 1×)
+Glutamax (Thermo Fisher Scientific Inc.) (final concentration: 1×)
+Penicillin/streptomycin (Thermo Fisher Scientific Inc.) (final concentration: 1×)
+Primocin (final concentration: 1 mg/ml)
+N-Acetyl-L-cysteine (final concentration: 1 mM)
+Wnt3 conditioned medium (50% v/v)
+RSPO1 conditioned medium (10% v/v)
+Noggin conditioned medium (10% v/v)
+EGF (final concentration: 50 ng/ml)
+Gastrin (final concentration: 10 nM)
+FGF10 (final concentration: 100 ng/mL)
+B27 (final concentration: 1×)
+Nicotinamide (final concentration: 10 mM)
+A83-01 (final concentration: 0.5u nM)

### 1-16. Preparation of Human Lung Cancer Cell Line Organoid

A pre-existing human lung cancer cell line (A549) was introduced from ATCC. In the present study, this cell line was introduced and then used at a passage number of 10 or less in experiments. The pre-existing human lung cancer cell line was transfected with luciferase gene in advance. The human lung cancer cell line, HUVECs, and hMSCs were inoculated onto a three-dimensional culture vessel (e.g., ELPLASIA plate from Kuraray Co., Ltd.) to reconstitute a human lung cancer cell line organoid. The human lung cancer cell line was inoculated at 3×103 cells on each well of a 96-well plate which was also inoculated with arbitrary numbers of HUVECs and hMSCs to reconstitute a cancer organoid. The mixing ratio of the cancer cells, HUVECs, and hMSCs was set to 10:0:0, 10:7:1 (Low hMSC), or 10:7:20 (High hMSC).

### 1-17. Method for Evaluating Radiation Sensitivity

A primary human pancreatic cancer organoid was subcutaneously transplanted to immunodeficient mice to form a xenograft. Then, the xenograft site was irradiated with a carbon beam (15 Gy). Changes in the size of the xenograft after the irradiation were measured to evaluate changes in tumor size.

1-18. Correlation of Drug Sensitivity of Primary Human Pancreatic Cancer Organoid with Patient Prognosis

Pancreatic cancer cells were separated from a surgically resected preparation of each pancreatic cancer patient and expanded culture was performed by the cyst culture method to obtain primary human pancreatic cancer cells. The pancreatic cancer cells thus subjected to expanded culture by the cyst culture method were confirmed to retain cell polarity even after the expanded culture. The obtained primary human pancreatic cancer cells were three-dimensionally cocultured with stromal cells (vascular endothelial cells (HUVECs, etc.) and mesenchymal cells (hMSCs, etc.)) to reconstitute a primary pancreatic cancer organoid. Its drug sensitivity was evaluated. The mixing ratio of these cells at the time of primary pancreatic cancer organoid preparation was 10:7:20. The number of specimens was 2.

### 2. Results

### 2-1. Discrepancy of Drug Sensitivity of Pre-Existing Human Pancreatic Cancer Cell Line Between In Vitro and In Vivo

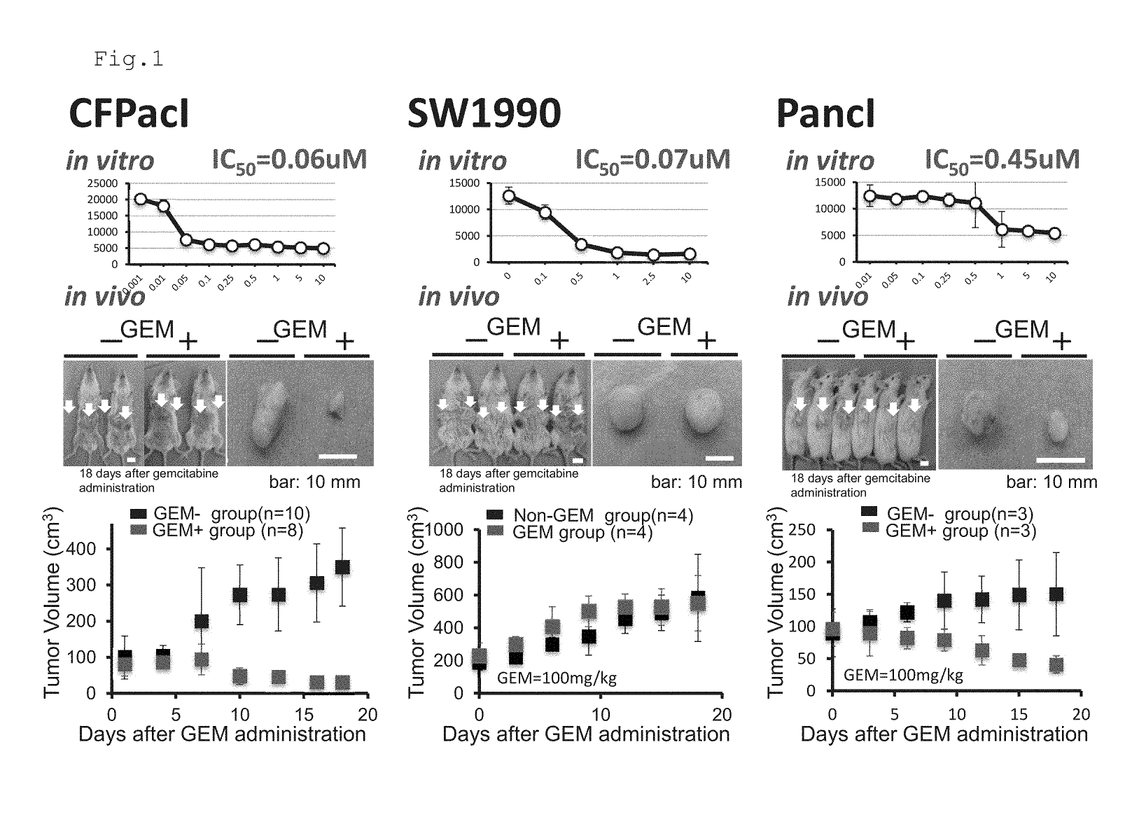

The in vitro drug sensitivity of a pre-existing human pancreatic cancer cell line CFPAC-1, PANC-1, or SW1990 was evaluated. 10⁻¹² to 10⁻³M GEM was added to the cells cultured for 24 hours, and IC50 was calculated from the number of cells surviving 72 hours after the addition. As a result, IC50 of CFPAC-1, PANC-1, or SW1990 was 0.03 μM, 0.7 μM, or 0.2 μM, respectively (upper panels of FIG. 1). On the other hand, the cancer cells were subcutaneously transplanted to NOD/Scid mice, and the xenograft formed was evaluated for its in vivo drug sensitivity by the administration of GEM at 100 mg/kg. As a result, tumor regression was found for CFPAC-1 and PANC-1 upon administration of GEM. On the other hand, no tumor regression was found for SW1990, and the tumor volume increased instead (lower panels of FIG. 1). Thus, it was revealed that: PANC-1 has relatively low drug sensitivity in vitro, but has high drug sensitivity in vivo; and SW1990 has relatively high drug sensitivity in vitro, but has low drug sensitivity in vivo. These results indicate that PANC-1 and SW1990 have discrepancy of drug sensitivity between in vitro and in vivo.

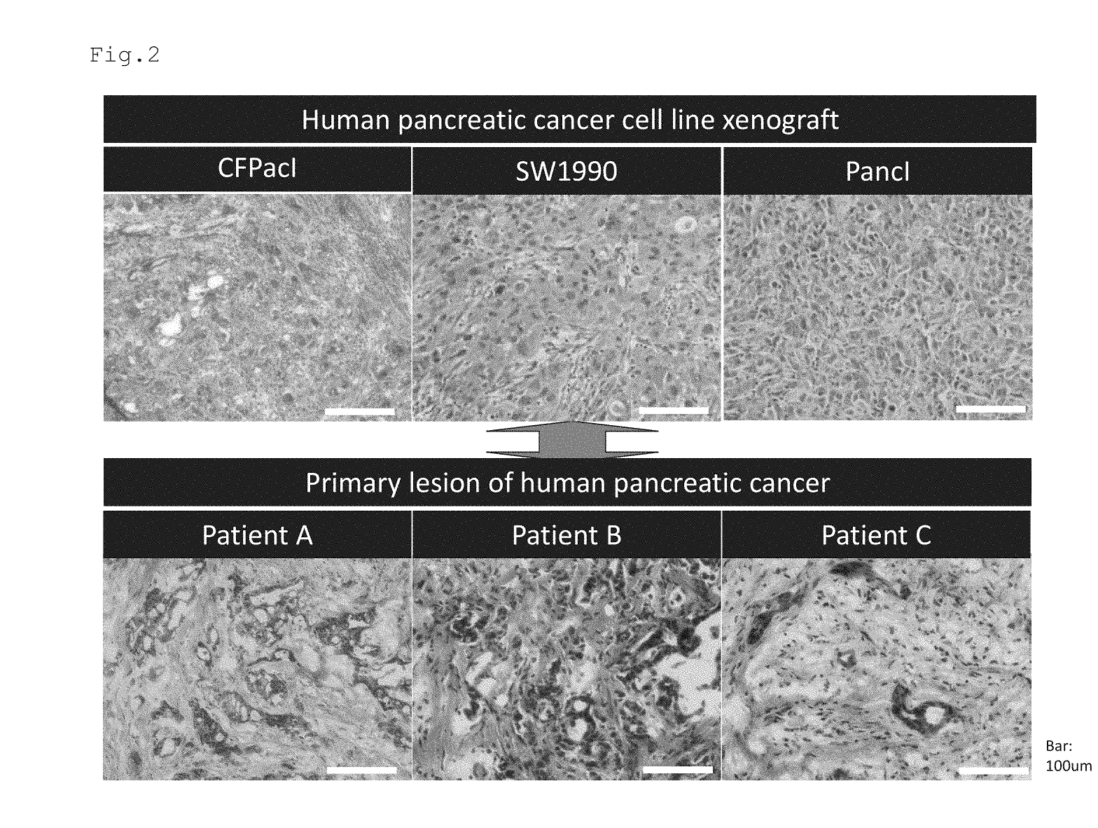

From the histological analysis of xenografts, discrepancy was confirmed to exist between the tissue images of a xenograft reconstituted from a pre-existing human pancreatic cancer cell line and a primary lesion of human pancreatic cancer. The xenograft reconstituted from a pre-existing human pancreatic cancer cell line was found to be free from rich stroma or a ductal structure, which is seen in the primary lesion of pancreatic cancer (FIG. 2).

### 2-2. Creation of Pancreatic Cancer Organoid Using Pre-Existing Human Pancreatic Cancer Cell Line

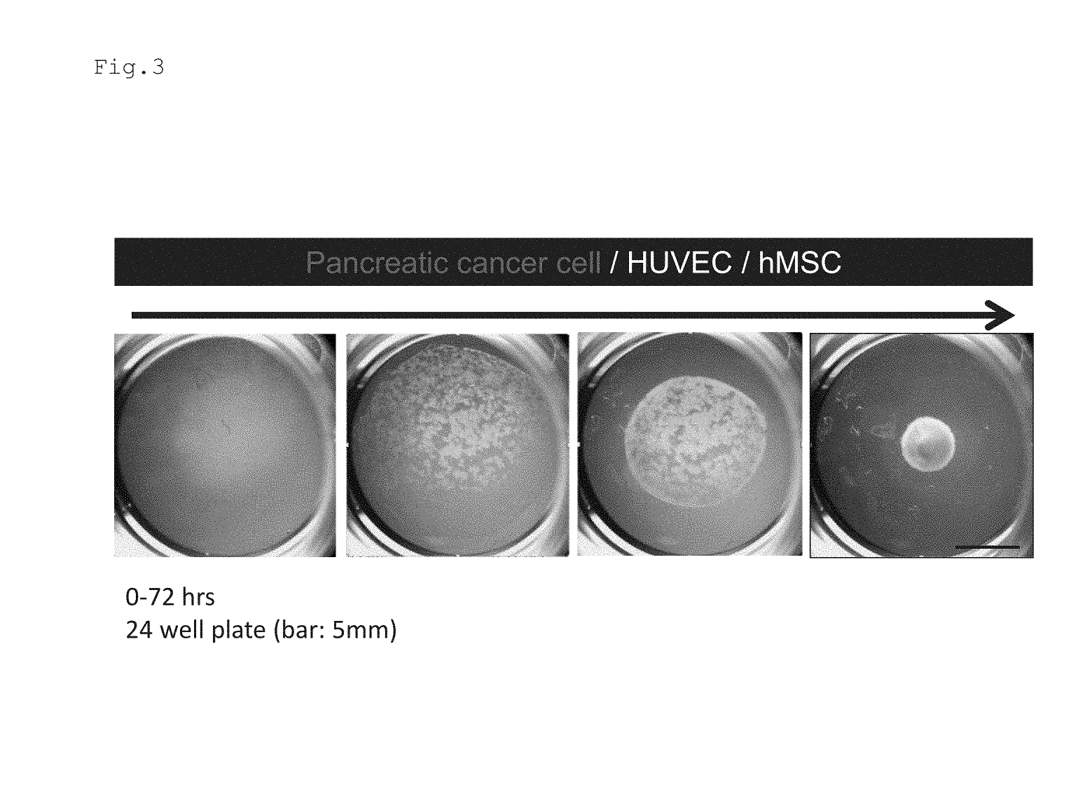

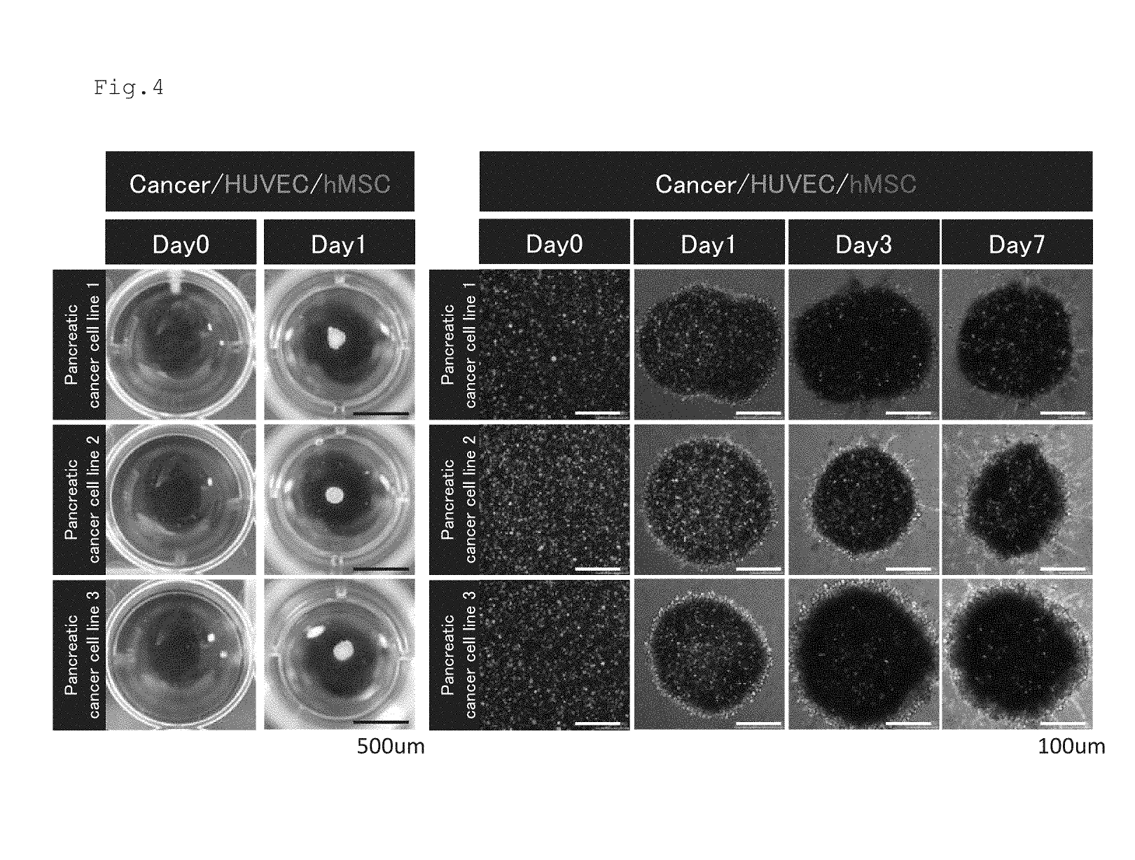

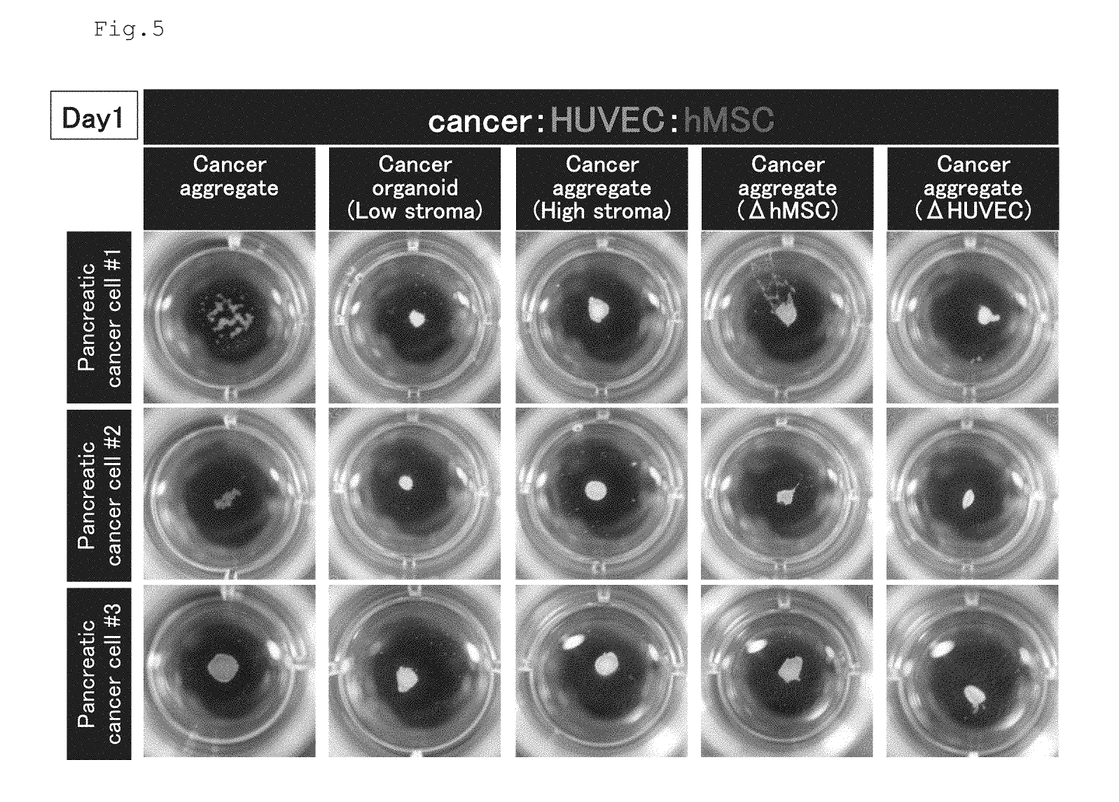

A pre-existing human pancreatic cancer cell line CFPAC-1, PANC-1, or SW1990 was cocultured with HUVECs and hMSCs. As a result, the autonomous aggregation of the cells was observed (FIG. 3). A pre-existing human pancreatic cancer cell line organoid composed of the pre-existing human pancreatic cancer cells, HUVECs, and hMSCs was formed at coculture day 1 using any of the cell lines (FIG. 4). The state of constitution of the organoid formed was observed by using as an index the expression of fluorescent reporters introduced in HUVECs and hMSCs. As a result, the three types of cells were confirmed to coexist homogeneously up to coculture day 1. However, the incidence of HUVECs decreased markedly at coculture day 3 or later. Therefore, in the present study, subsequent experiments were conducted targeting an organoid of coculture day 1. The condition for mixing HUVECs and hMSCs for organoid formation was studied using each individual pre-existing human pancreatic cancer cell line. As a result, it was confirmed that an organoid with a high mixing ratio of hMSCs aggregates strongly, whereas an organoid free from hMSCs or with a low mixing ratio of hMSCs aggregates weakly, is physically fragile, and collapses easily (FIG. 5).

### 2-3. Histological Analysis of Pre-Existing Human Pancreatic Cancer Cell Line Organoid-Derived Xenograft

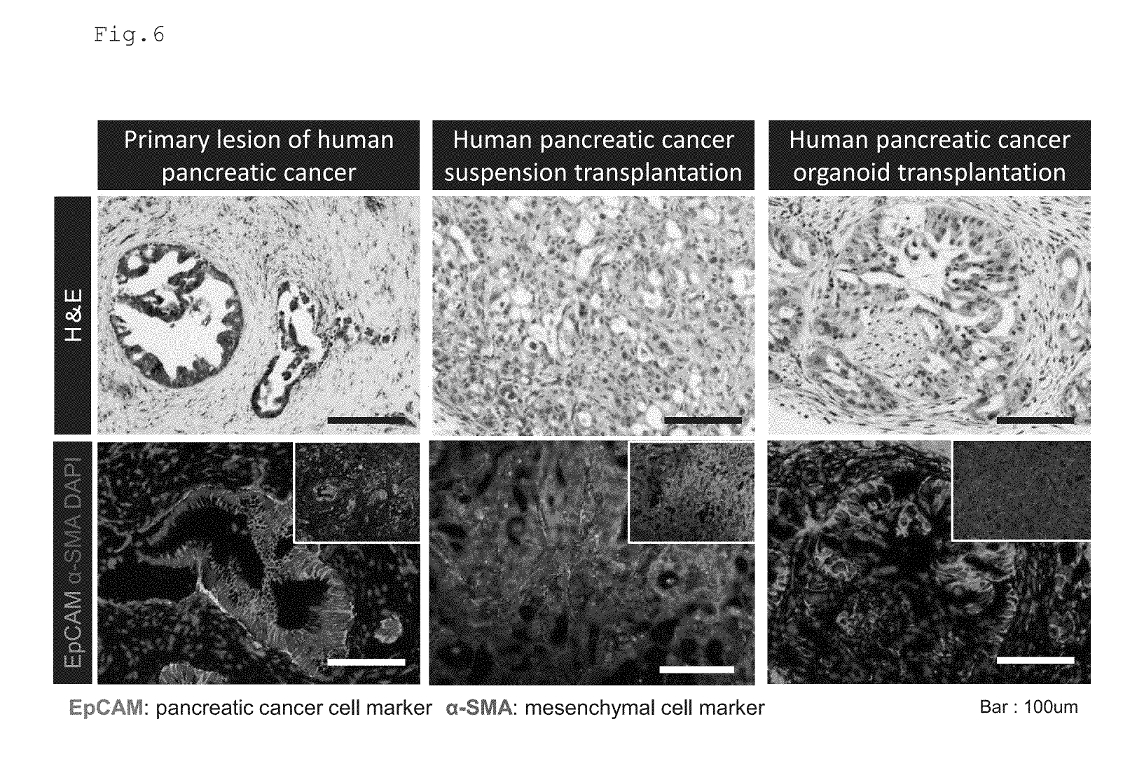

A pre-existing human pancreatic cancer cell line organoid was transplanted to NOD/Scid mice, followed by the analysis of reconstituted human pancreatic cancer tissue. As a result, rich stroma as well as a ductal structure were confirmed in the organoid transplantation group. On the other hand, no ductal structure was observed in a group transplanted with a pre-existing human pancreatic cancer cell line alone (FIG. 6). Next, organoids were prepared at various cell mixing ratios, and tissue images of respective xenografts reconstituted from these organoids were compared. In order to evaluate the states of reconstitution of stroma and blood vessels in the reconstituted tissue, the expression of a mesenchymal cell marker αSMA was studied. The proportion of αSMA-positive cells was evaluated by immunohistochemical staining and compared with a primary lesion. The graphs of the figure show the αSMA-positive cells, Sirius red-positive area, and Azan stain-positive area of a xenograft formed after transplantation of a suspension of only pancreatic cancer, a pancreatic cancer organoid having hMSCs mixed in a small number (Low hMSC), or a pancreatic cancer organoid having hMSCs mixed in a large number (High hMSC) (FIG. 7). Also, a hyaluronic acid-positive area, a collagen fiber area, and a tenascin-C-positive area are shown (FIG. 8). The collagen fiber area was evaluated under a polarizing microscope. Red color mainly depicts type I collagen fiber, and green color mainly depicts type III collagen fiber. Results of quantification are shown in the lower panels. The error bar represents standard deviation. A xenograft reconstituted from a pancreatic cancer organoid with high incidence of hMSCs exhibited features closely analogous to a primary lesion of human pancreatic cancer.

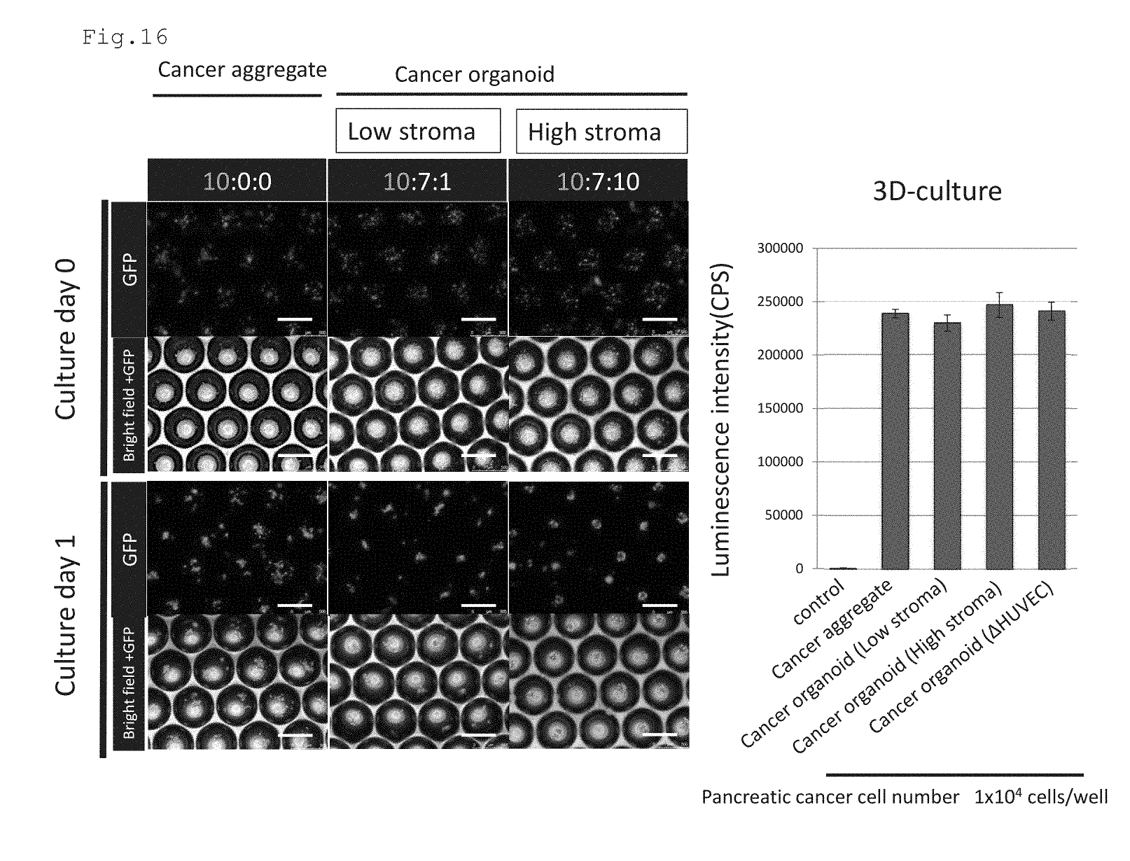

### 2-4. Construction of Cancer Cell-Specific Cell Detection Method Targeting Cancer Organoid (FIG. 16)

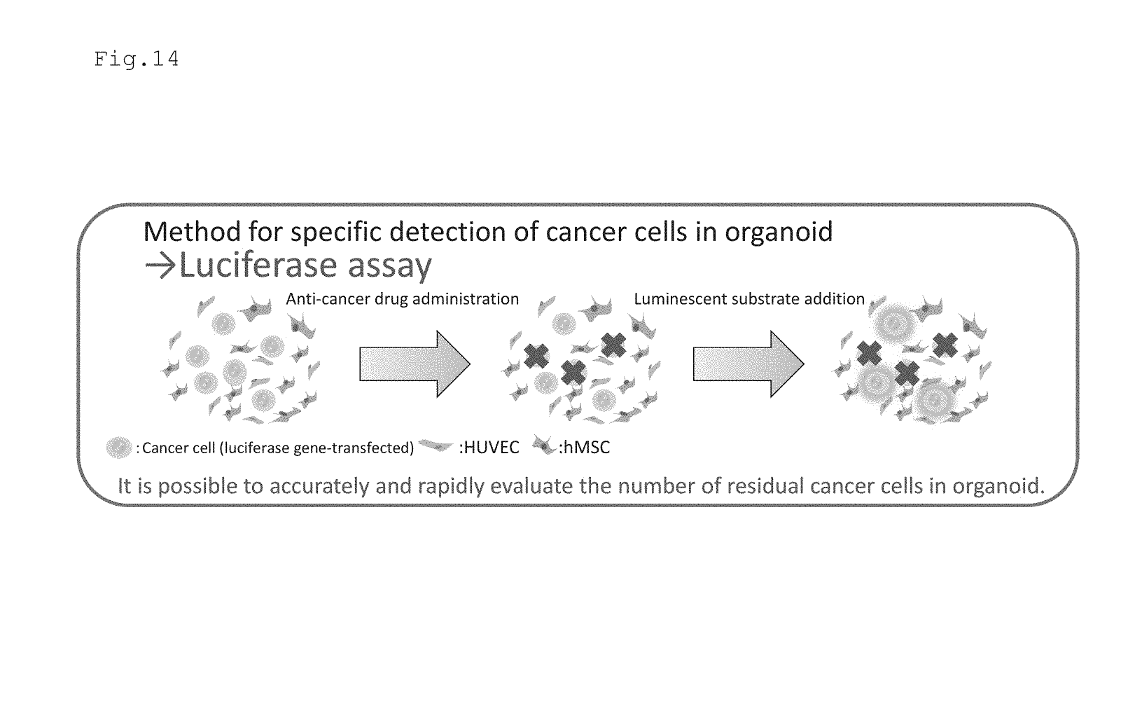

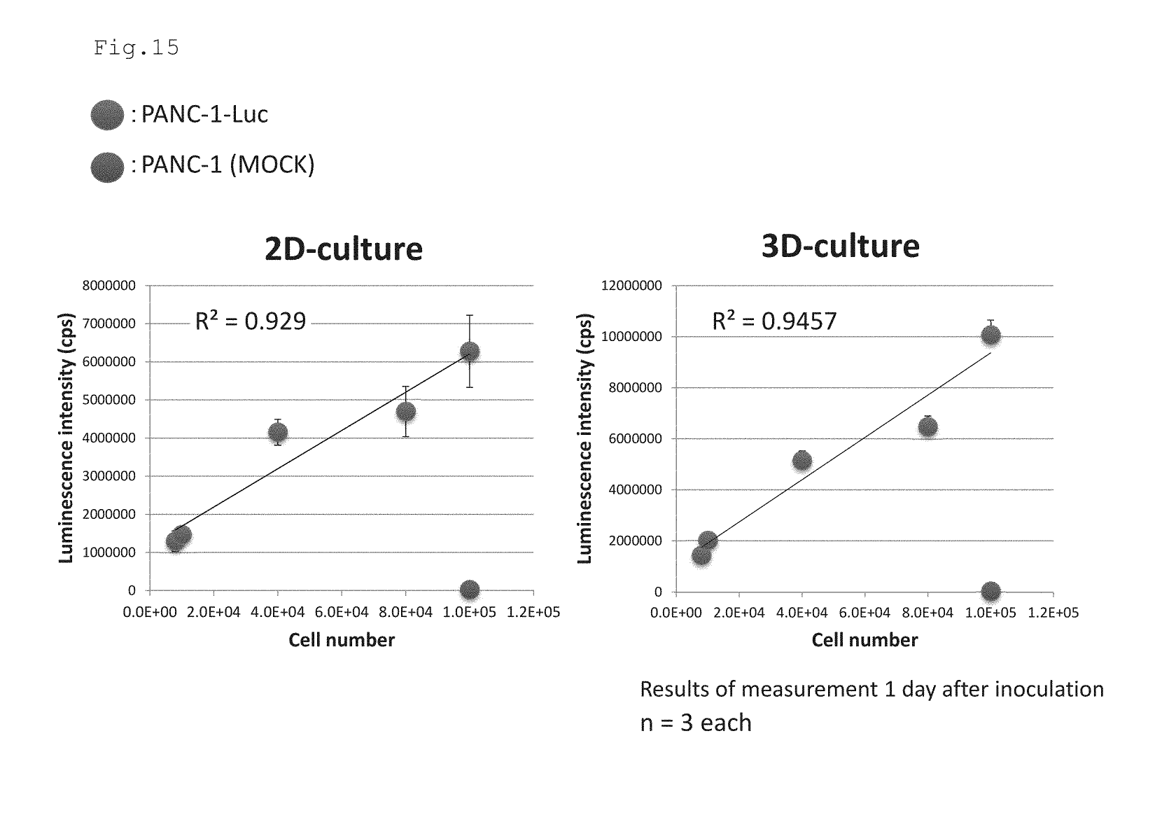

In order to evaluate the drug sensitivity of cancer cells with high accuracy, an approach for quantitatively evaluating only the number of cancer cells within a cancer organoid was studied (FIG. 14). Luciferase gene-transfected cancer cells (CFPAC-1, PANC-1, or CAPAN-2; typically, CFPAC-1) were established, and a cancer organoid was reconstituted. Then, a luminescent substrate was added thereto, and the luminescence intensity of each well was measured using a luminescence plate reader. The luciferase gene-transfected cancer cells were inoculated at various cell numbers onto a multi-well plate, followed by luciferase assay. As a result, the luminescence intensity was confirmed to be proportional to the cell number (FIG. 15). It was also confirmed that the luciferase activity in the cancer organoid is not influenced by a stromal cell number (FIG. 16).

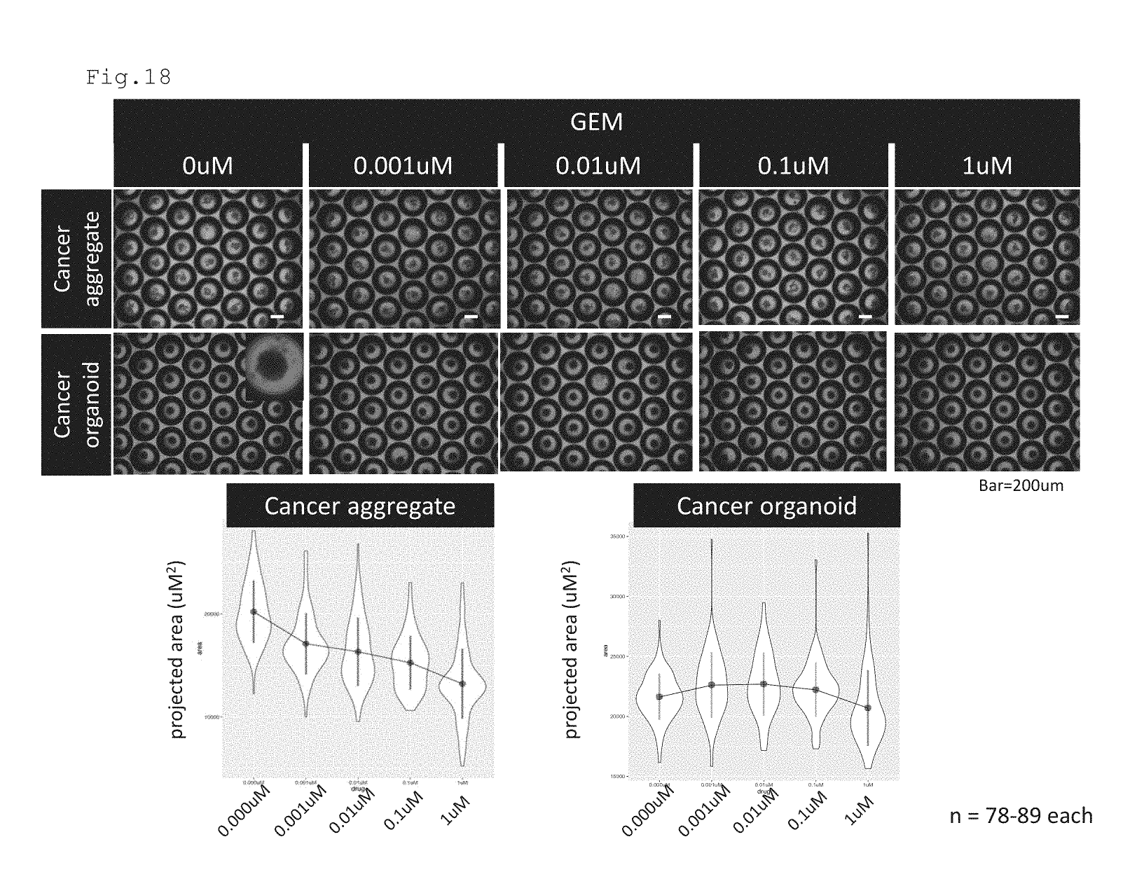

### 2-5. Changes in the Size of Organoid After Gemcitabine Administration (FIG. 18)

Response after anti-cancer drug administration was evaluated with a cancer organoid size used as an index (FIG. 18). Images of a cancer organoid at 72 hours after anti-cancer drug administration were captured, and the area of the cancer organoid was calculated by image analysis (software manufactured by GE Healthcare Japan Corp. was used). The image information on the organoid was confirmed to enable convenient evaluation oft drug sensitivity.

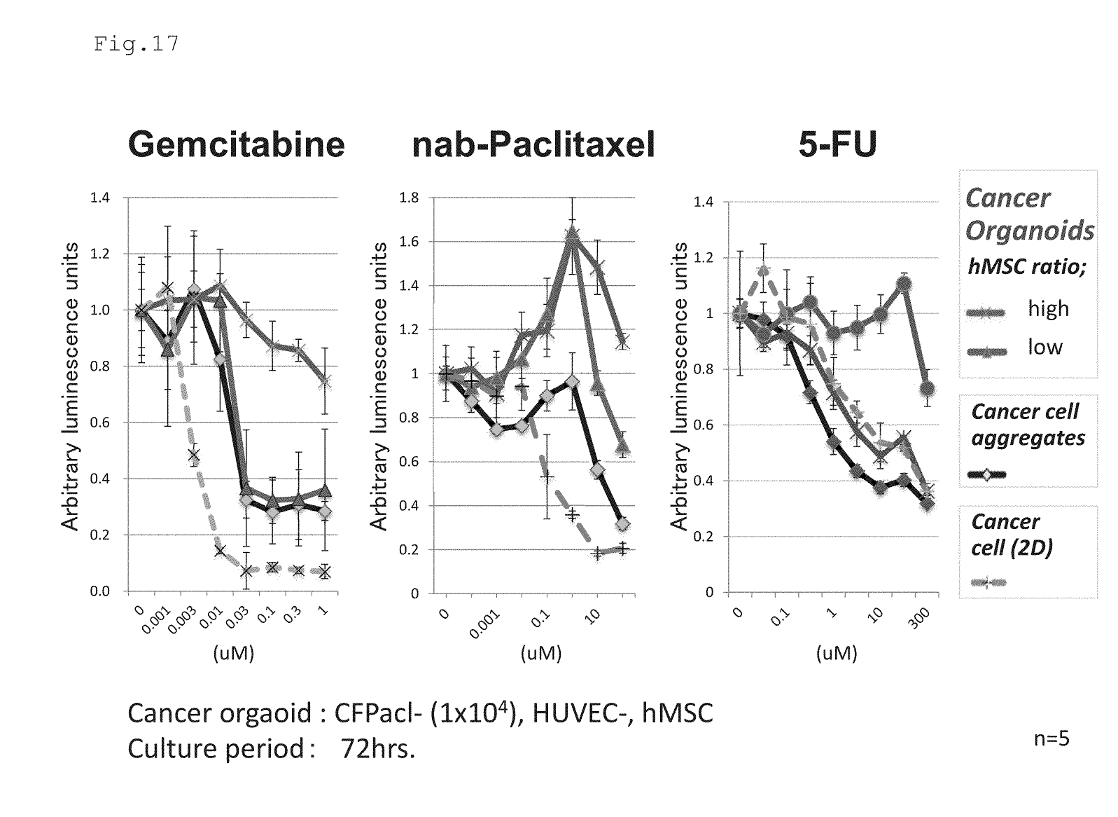

### 2-6 Stroma-Rich Cancer Organoids Exhibit Anti-Cancer Drug Resistance In Vitro (FIG. 17)

A pancreatic cancer organoid was reconstituted from luciferase gene-transfected pancreatic cancer cells and stromal cells and evaluated for its sensitivity for a pancreatic cancer therapeutic (anti-cancer drug) (FIG. 17). The gray dotted line depicts the drug sensitivity of two-dimensionally cultured cancer cells. The black solid line depicts the drug sensitivity of three-dimensionally cultured cancer cells (cancer cell aggregate). The red solid line (cancer organoid containing stromal cells with high incidence) and the blue solid line (cancer organoid having low incidence of presence of stromal cells) depict the drug sensitivity of a three-dimensionally cultured cancer organoid. The cancer organoid containing stromal cells with high incidence is confirmed to exhibit high drug resistance to any of the drugs.

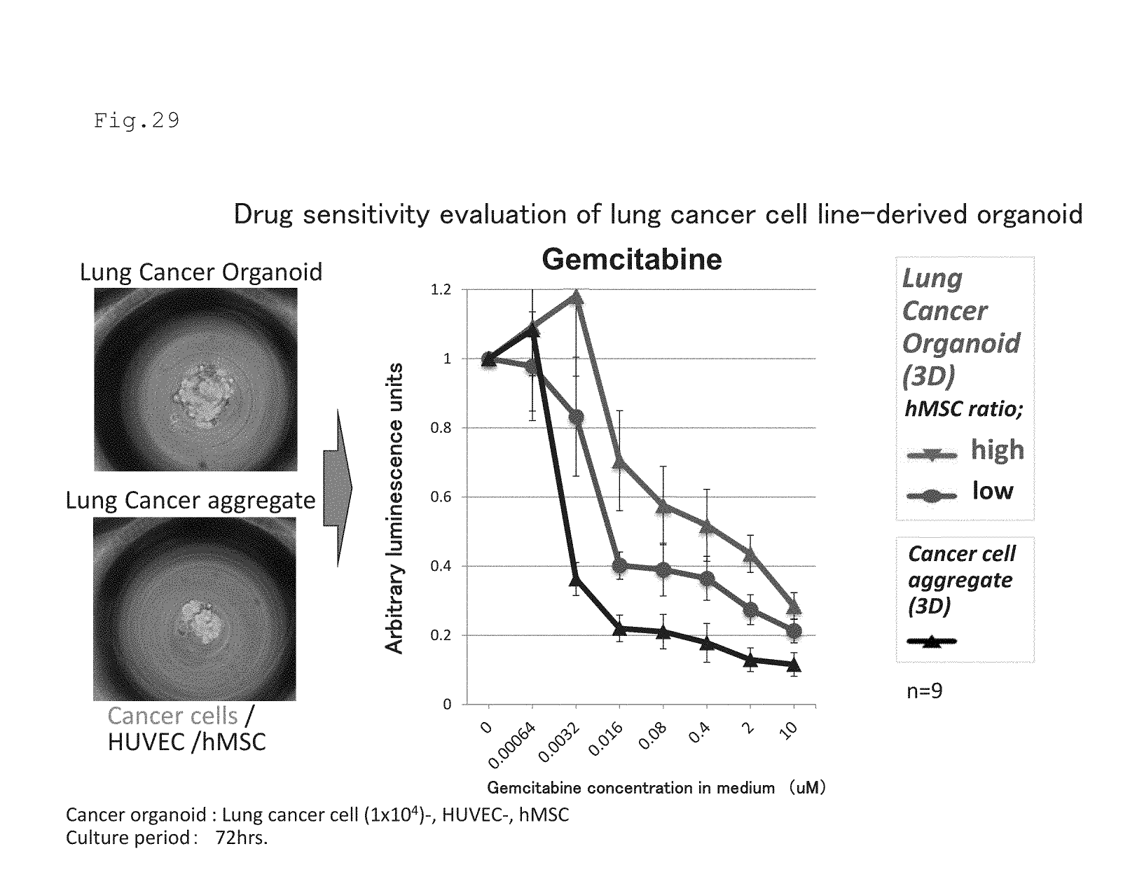

### 2-7 Creation of Pre-Existing Human Lung Cancer Cell Line Organoid (FIG. 29)

A lung cancer organoid prepared three-dimensionally using a luciferase gene and EGFP expressing human lung cancer cell line (A549 cells), HUVECs, and hMSCs, and a three-dimensional aggregate composed of only pancreatic cancer cells were evaluated for their in vitro drug sensitivity. The left diagrams show a fluorescence phase-contrast microscope image of the lung cancer organoid. In the graph of the right diagram, the ordinate depicts the amount of luciferase activity of the lung cancer cells, and the abscissa depicts an anti-cancer drug (gemcitabine) concentration in a medium. The lung cancer cell aggregate exhibits high sensitivity for gemcitabine. On the other hand, the pancreatic cancer organoid culture groups have lower drug sensitivity for gemcitabine. Among the pancreatic cancer organoid groups, the group involving hMSCs and HUVECs with high incidence (High stroma group) has even lower sensitivity for the anti-cancer drug.

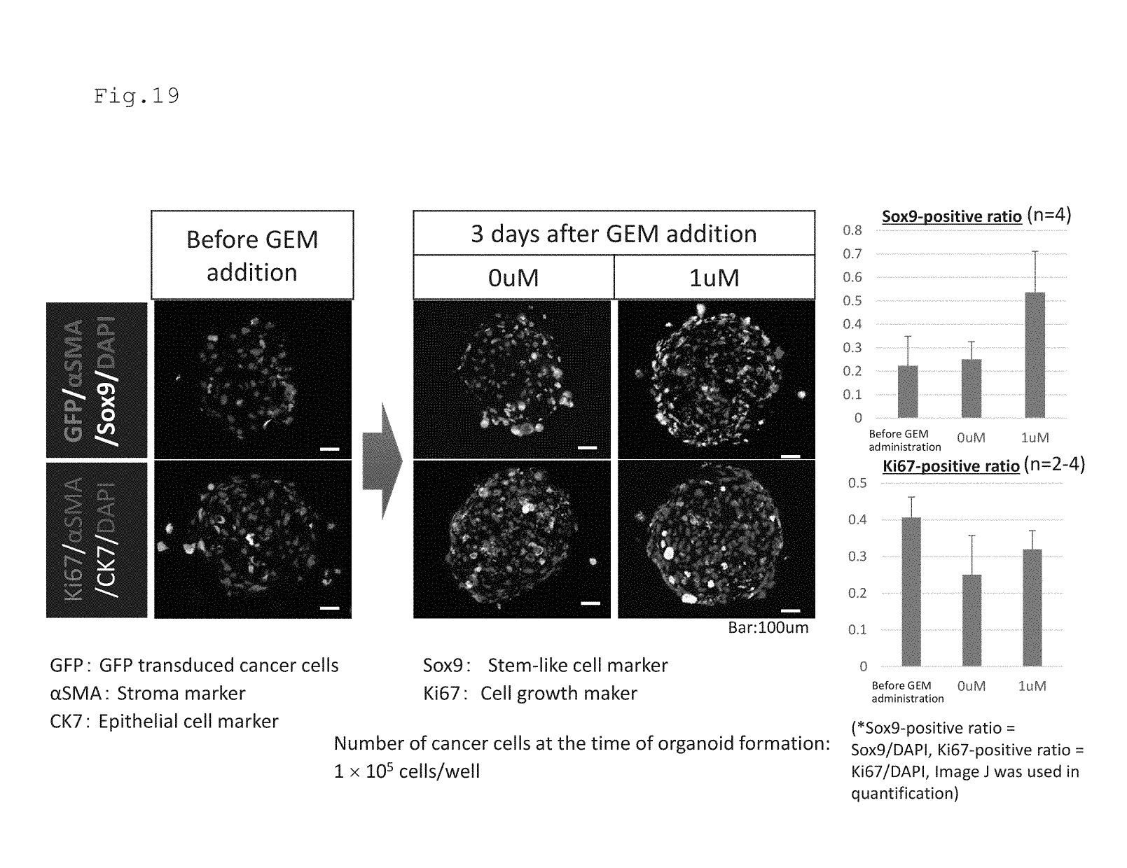

### 2-8. Pancreatic Cancer Organoids are Useful in Evaluation of Pancreatic Cancer Stem Cells (FIG. 19)

The properties of cancer cells remaining after anti-cancer drug addition and stromal cells within a cancer organoid were evaluated. EGFP gene-transfected cancer cells (typically, CFPAC-1) were established, and a cancer organoid was reconstituted (the cancer cell:HUVEC:hMSC ratio is, for example, 10:7:10 to 10:7:20). Then, the cancer organoid was cultured for 72 hours in a medium containing 1 uM gemcitabine. GFP-positive and Sox9-positive cancer stem cells are confirmed to remain in the inside of the cancer organoid as a result of the addition of the anti-cancer drug (right diagrams of upper panels).

2-9. Drug Sensitivity of Xenograft Derived from a Pre-Existing Human Pancreatic Cancer Cell Line Organoid

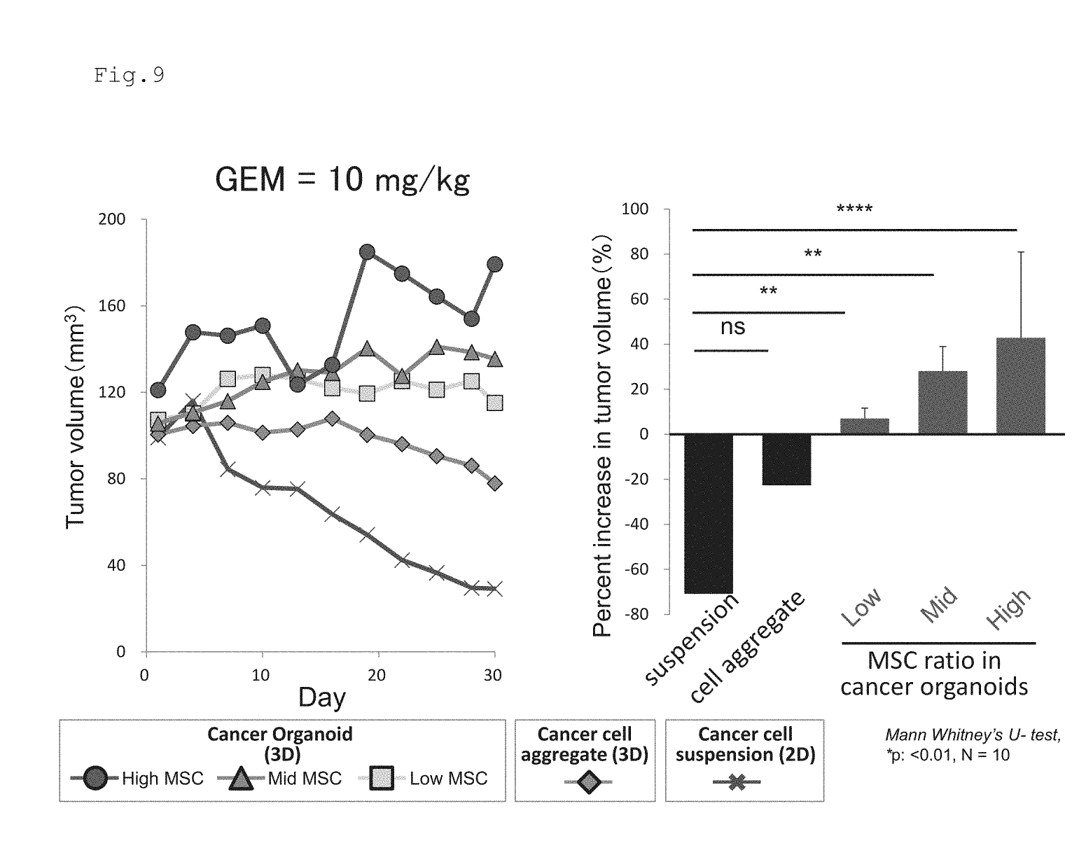

The in vivo drug sensitivity of a xenograft reconstituted after transplantation of each organoid was evaluated using a typical pancreatic cancer therapeutic Gemzar (gemcitabine: GEM). A pre-existing human pancreatic cancer cell line organoid prepared by the three-dimensional coculture of a pre-existing human pancreatic cancer cell line, HUVECs, and hMSCs was subcutaneously transplanted to NOD/Scid mice. Then, the administration of GEM (e.g., 10 mg/kg) was started from the point in time when the tumor volume exceeded 100 mm³. A non-GEM-administration group (0 mg/kg) given only physiological saline was set as a control group. GEM was administered once every three days for 30 days with reference made to a treatment regimen for human pancreatic cancer. A xenograft was recovered at GEM administration day 30 and analyzed histologically. In all of the transplantation groups, the volume of the xenograft of the non-GEM-administration group increased as the days went by, whereas the increase in the volume of the xenografts of the GEM administration groups (e.g., 10 mg/kg) was suppressed (FIG. 9). From the comparison of the tumor volumes of the GEM administration groups (e.g., 10 mg/kg), a xenograft formed from a pancreatic cancer organoid having hMSCs mixed in a large number (High hMSC) exhibited no regression and increased in volume, whereas xenografts formed from organoids of the other groups exhibited regression (FIG. 9). As seen from these results, a stroma-rich xenograft formed from an organoid with a cell mixing ratio having a large proportion of hMSCs had reduced drug sensitivity.

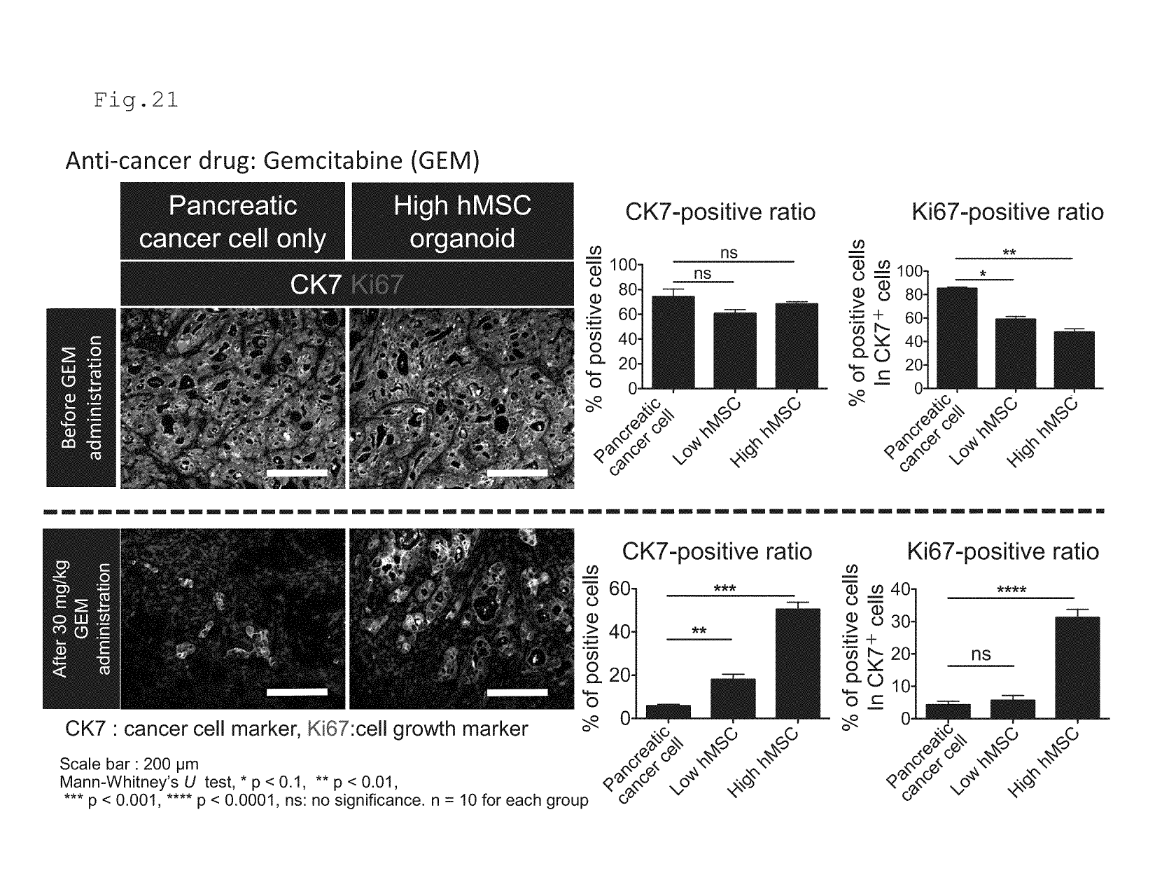

### 2-10 Pancreatic Cancer Organoids Exhibit Anti-Cancer Drug Resistance In Vivo (FIG. 21)

A pre-existing human pancreatic cancer cell line was transplanted at 2×10⁵ cells to immunodeficient mice. After a xenograft reached 100 mm³, gemcitabine was administered thereto once every three days. An immunostaining image of the xenograft recovered 1 month after the start of GEM administration is shown. The inside of the xenograft after the GEM administration exhibits a structure similar to human pancreatic ductal adenocarcinoma. This figure shows the expression of cytokeratin 7 (CK-7, white) and Ki-67 (red). The upper panels show a tissue image before gemcitabine administration, and the lower panels show a tissue image after gemcitabine administration. A pancreatic cancer organoid-derived xenograft has high incidence of Ki67-positive cells after anti-cancer drug administration and exhibits strong resistance to the anti-cancer drug (FIG. 21).

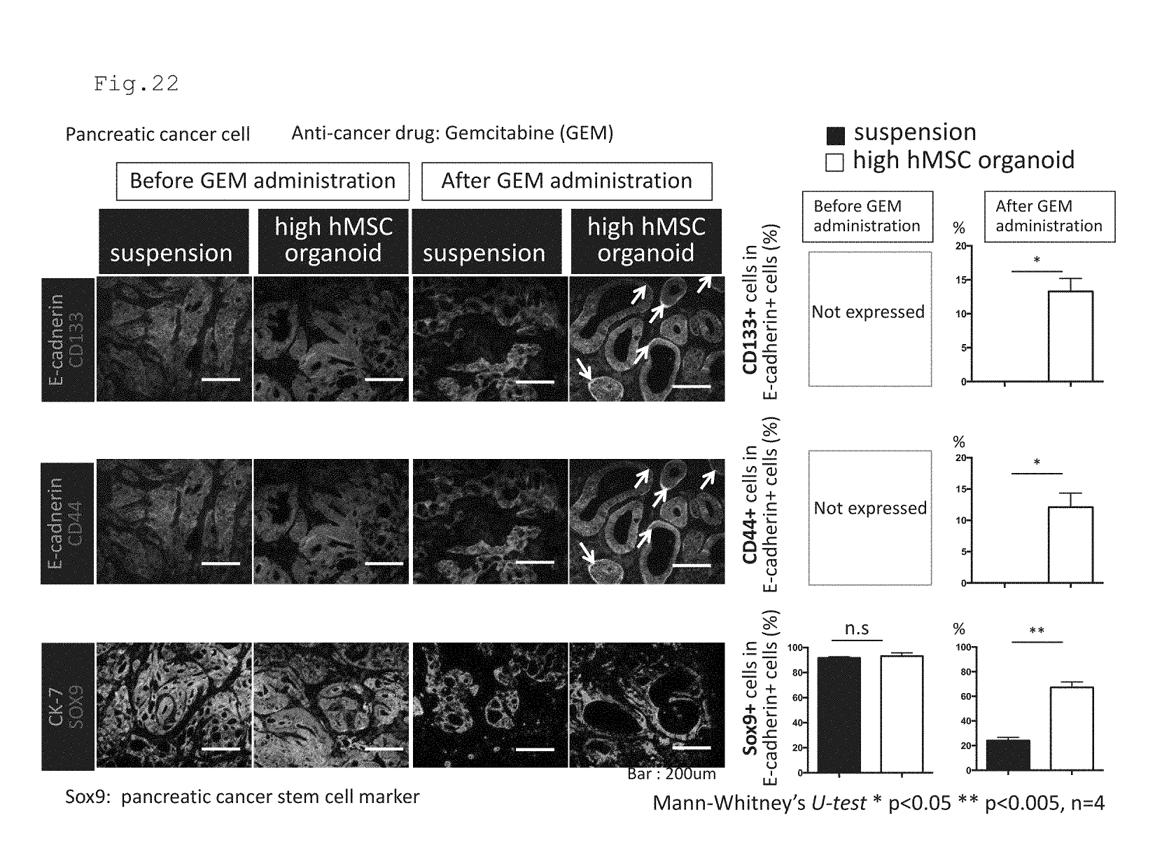

### 2-11 Pancreatic Cancer Organoid-Derived Xenografts Enable Evaluation of Residual Cancer Stem Cells (FIG. 22)

The expression of cancer stem cell markers (CD133, CD44, and Sox9) was studied in pancreatic cancer tissue remaining after anti-cancer drug administration. As a result, it was revealed that pancreatic cancer cells expressing these molecules remain in a pancreatic cancer organoid-derived xenograft (FIG. 22). On the other hand, after anti-cancer drug administration, cells positive for these markers were substantially absent from a xenograft formed after the transplantation of pancreatic cancer suspension (FIG. 22). A pancreatic cancer organoid-derived xenograft was confirmed to be beneficial to the evaluation of cancer stem cells.

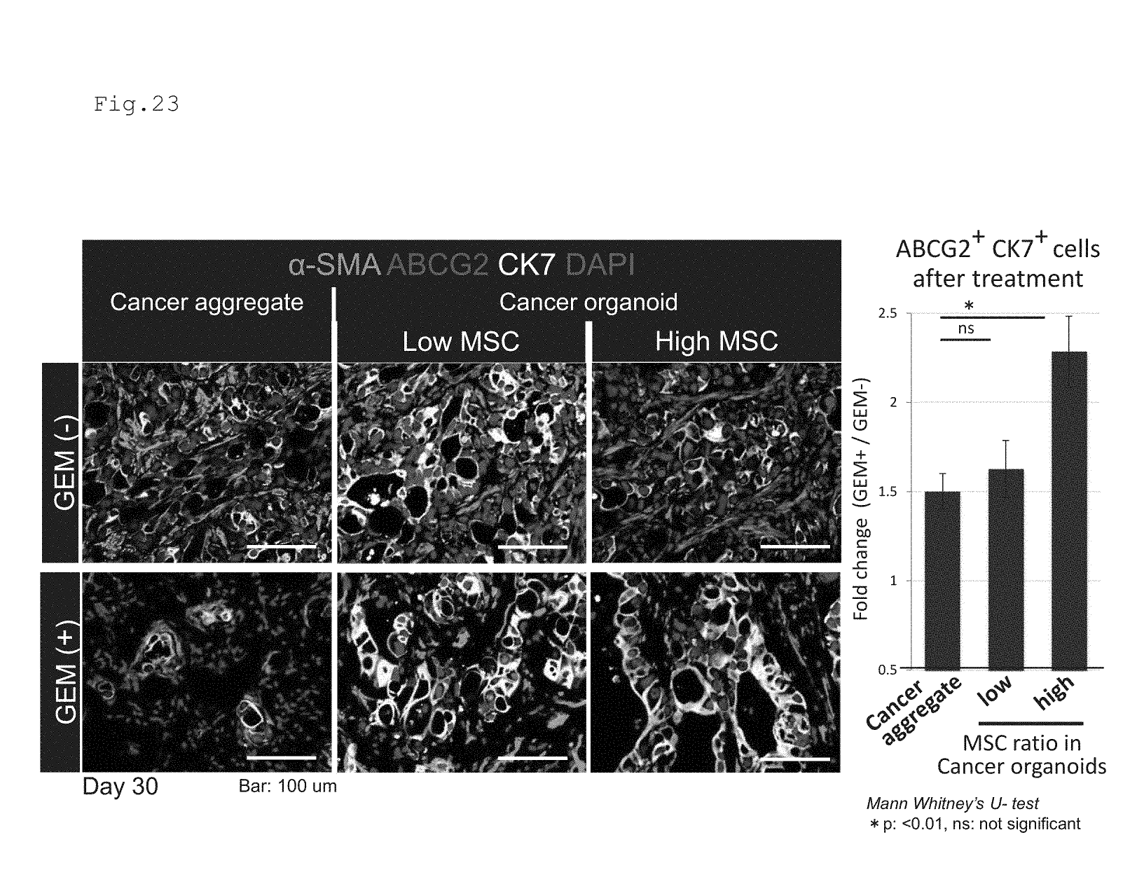

### 2-12 Expression of Multidrug Resistance Transporter is Enhanced in Cancer Organoid-Derived Xenografts (FIG. 23)

A pancreatic cancer cell line was transplanted to immunodeficient mice. Then, gemcitabine administration was started from the point in time when a xenograft reached 100 mm³. Results of analyzing tissue recovered at gemcitabine administration day 30 are shown. A staining image of a multidrug resistance transporter (ABCG2) is indicated by red color, a staining image of cytokeratin 7 (CK7) by white color, αSMA staining results by green color, and a DAPI staining image by blue color. Pancreatic cancer cells expressing ABCG2 are confirmed to remain in a cancer organoid transplantation group after gemcitabine administration.

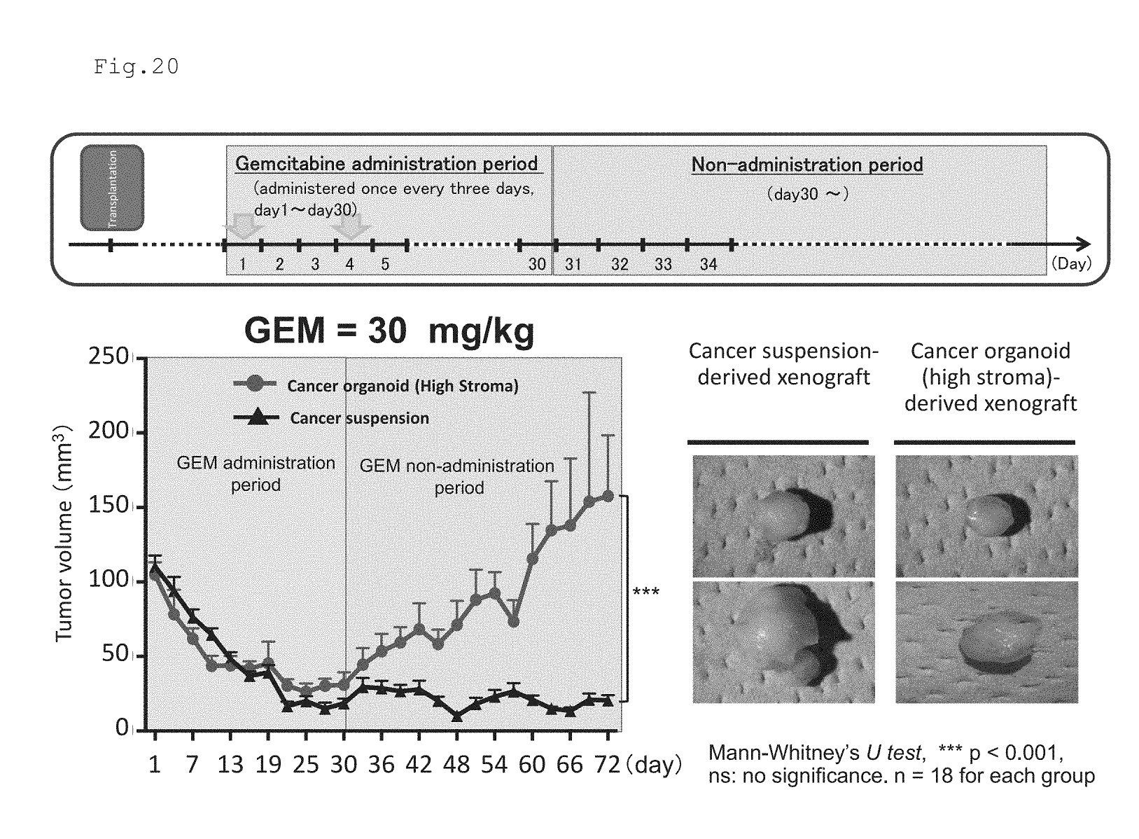

### 2-13 Stroma-Rich Xenografts Increase in Volume After Discontinuation of GEM Administration (FIG. 20)

After cancer organoid (CFPAC-1-derived) transplantation, gemcitabine was administered (30 mg/kg) for 30 days. Then, the gemcitabine administration was discontinued. Subsequent variation in tumor size was confirmed. A suspension transplantation group treated with gemcitabine had a constant tumor size even after the discontinuation of the administration. By contrast, the tumor size of the pancreatic cancer organoid transplantation group treated with gemcitabine increased markedly after the discontinuation of the administration. In short, a pancreatic cancer organoid was confirmed to be able to reproduce tumor recurrence after discontinuation of anti-cancer drug administration.

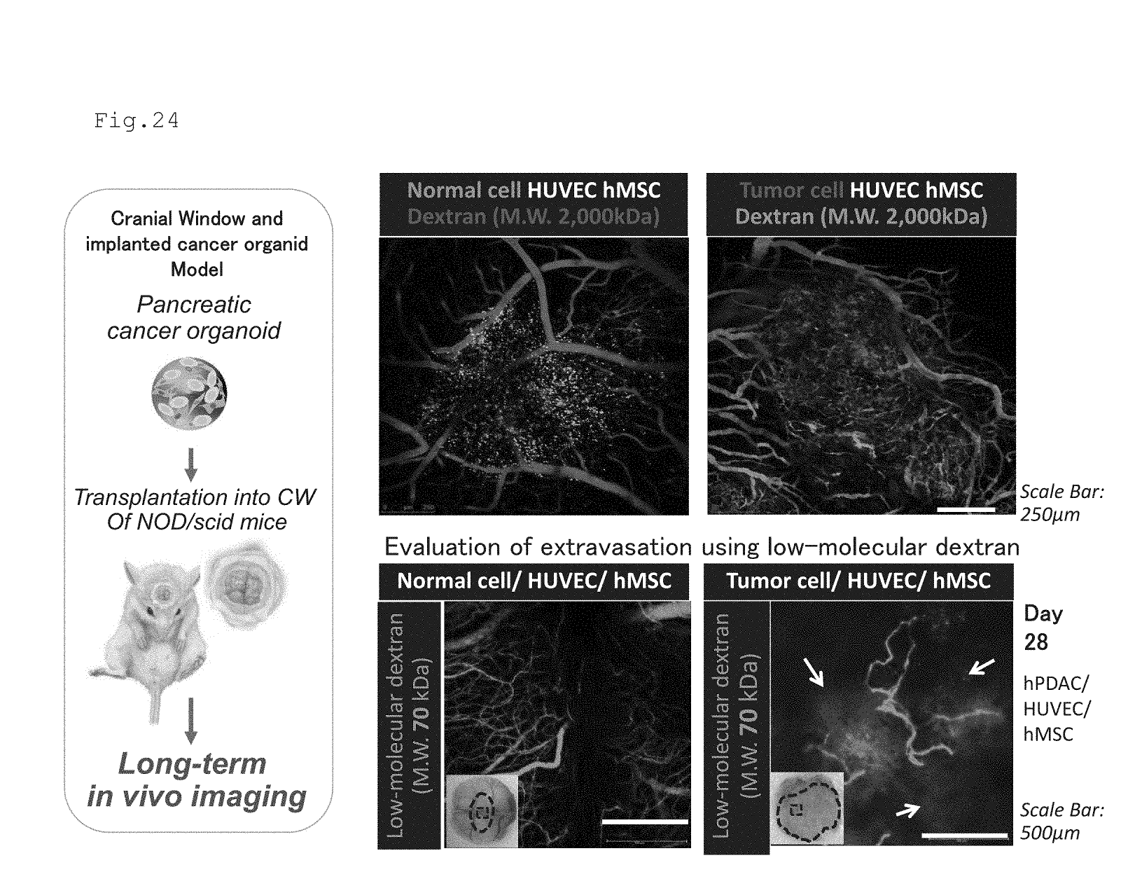

### 2-14 Reconstitution of Human Pancreatic Cancer Xenograft Having Blood Vessels Within Cranial Window (FIG. 24)

A pancreatic cancer organoid (EGFP-incorporating pancreatic cancer cell (CFPAC-1-derived) number: 2×10⁵ cells) was transplanted into a cranial window prepared in the head of an immunodeficient mouse. A cranial window image 28 days after the transplantation is shown (FIG. 24). The network construction of HUVECs is observed immediately after pancreatic cancer organoid transplantation. In order to visualize the vascular network within the cranial window, high-molecular-weight fluorescent dextran (M.W. 2,000 kDa) was injected from the mouse tail vein, and images were captured within 15 minutes. The upper panels of the right diagrams show an image of cancer cells expressing the fluorescent gene and an image of blood vessels labeled with high-molecular-weight fluorescent dextran. A tumor vessel structure that exhibits heterogeneous and excessive branches is confirmed within a xenograft formed after pancreatic cancer organoid transplantation. The extravasation of low-molecular dextran is further detected in the xenograft after pancreatic cancer organoid transplantation. Reference for the cranial window preparation method: Takebe T, Taniguchi H et al., Nature. 2013 Jul. 25; 499 (7459): 481-4.

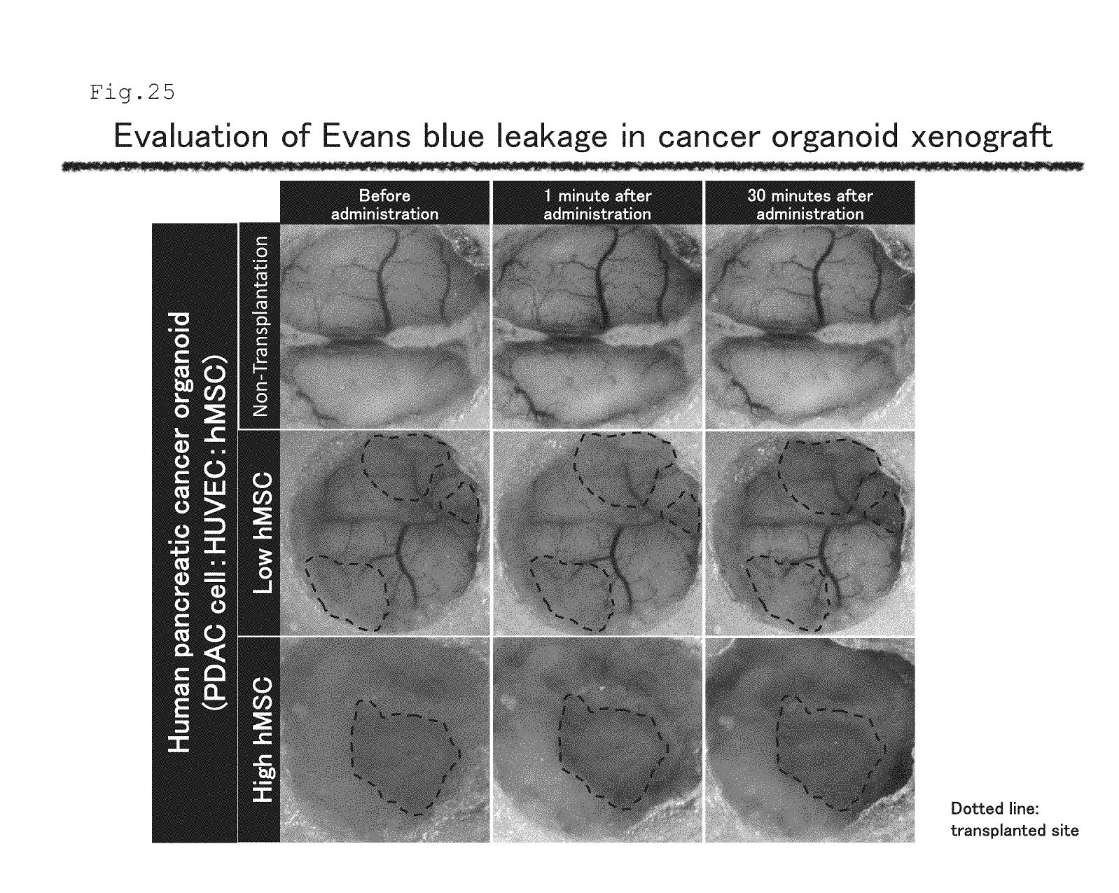

### 2-15 Evaluation of Tumor Vessel Within Xenograft (Evaluation of Leakiness) (FIG. 25)

A pancreatic cancer organoid (pancreatic cancer cell (CFPAC-1) number: 2.0×10⁵ cells) was transplanted into a cranial window, and the leakiness of blood vessels constructed within the cranial window was evaluated. After administration of 0.5% Evans blue containing physiological saline from the tail vein, the leakage of Evans blue to the periphery of the blood vessels within the cranial window was evaluated. A non-transplantation group had a small amount of residual Evans blue 30 minutes after the administration. On the other hand, residual Evans blue is confirmed over a prolonged periodin the cancer organoid transplantation group. Blood vessels formed after cancer organoid transplantation are confirmed to have a tendency for leakage.

The studies described above have established in vitro and in vivo drug evaluation systems using cancer organoids e. The drug sensitivity of cancer cells can be evaluated under physiological conditions by using these drug evaluation systems using cancer organoids. By evaluating the drug sensitivity of cancer cells using such an organoid with a cancer microenvironment, it would be possible to evaluate the drug resistance of cancer cells in an accurate way.

This holds anticipation for applications to the development of novel cancer therapeutics. Furthermore, the cancer organoid can be applied to drug evaluation using primary cancer cells separated from a clinical specimen such as a surgically resected specimen. Information for selecting a treatment method adapted for each cancer patient can be provided by reconstituting a cancer organoid having a cancer microenvironment from a clinical specimen and conducting drug evaluation. In addition, ripple effects toward the development of biomarkers for stratification of cancers are also expected by preparing cancer organoids using cancer cells separated from various patients, and conducting stratification with sensitivity for various drugs used as an index.

Meanwhile, this approach is considered to be also beneficial as an analytical tool for basic research such as the analysis of intercellular interaction. The application of this approach is also considered to enable reproduction of the interaction of cancer cells with other cell components involved in the cancer microenvironment (e.g., macrophages and neurons).

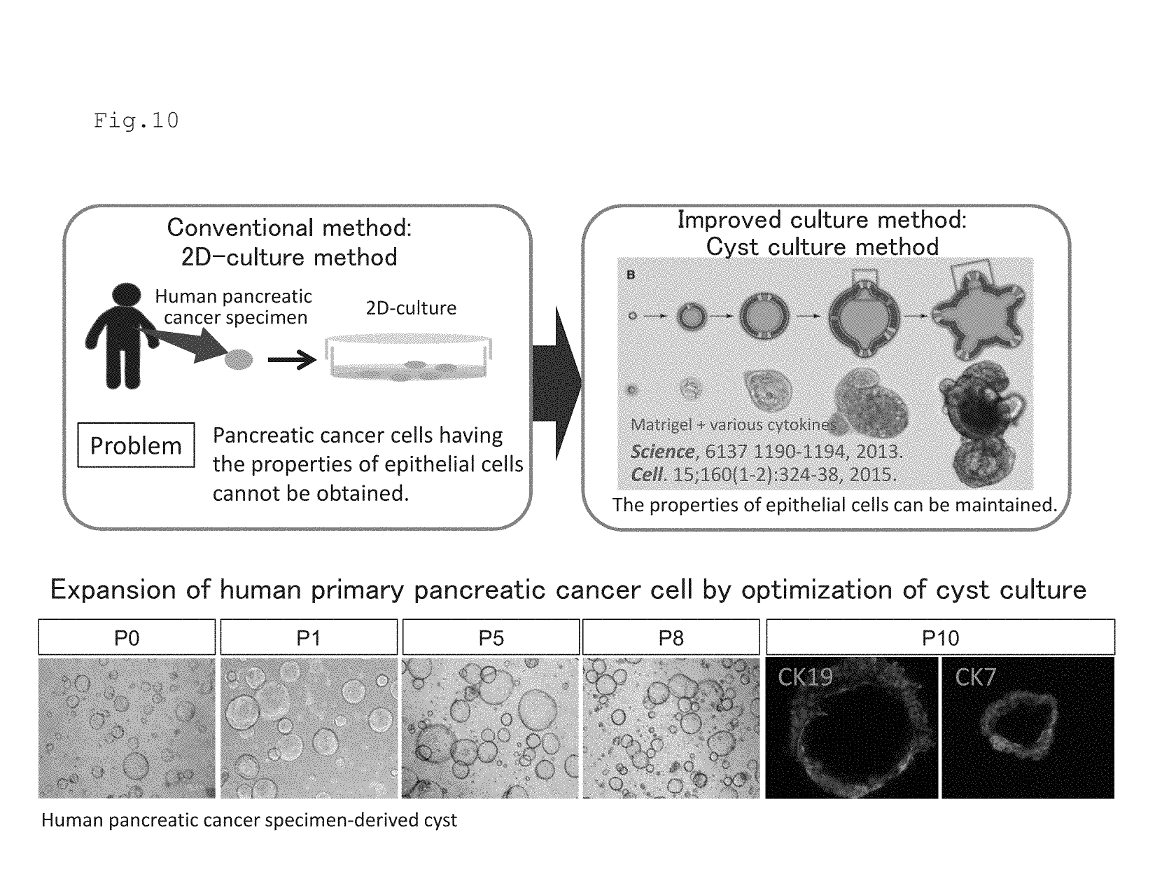

### 2-16 Reconstitution of Primary Organoid of Human Pancreatic Cancer (FIG. 10)

Pancreatic cancer cells were separated from surgically resected preparations of pancreatic cancer patients under informed consent. The pancreatic cancer cells were subjected to expanded culture using the cyst culture method. The pancreatic cancer cells obtained by expanded culture are confirmed to retain cell polarity (FIG. 10).

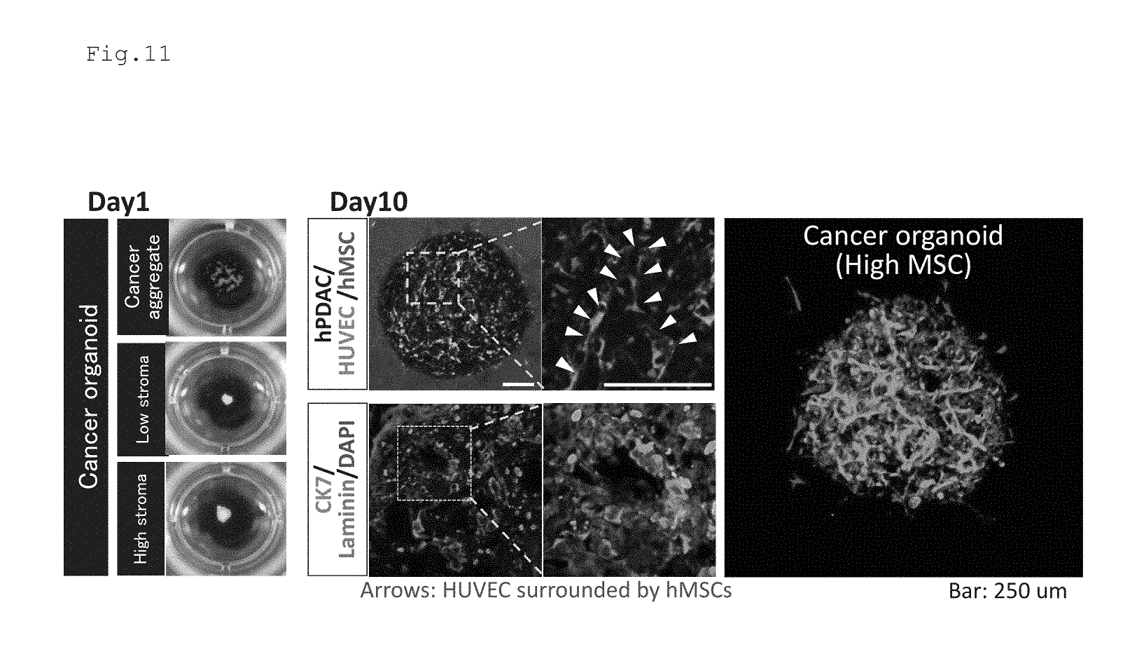

2-17 Pancreatic Duct-Like Structure Reconstituted within Primary Organoid of Human Pancreatic Cancer (FIG. 11)

Human primary pancreatic cancer cells, HUVECs, and hMSCs were three-dimensionally cocultured in vitro. A tissue image of the obtained primary pancreatic cancer organoid is shown (FIG. 11). The left diagrams show morphology at culture day 1. The right diagrams show morphology at culture day 10. Tissue closely analogous to a primary lesion, such as a pancreatic duct-like structure or a blood vessel-like structure, is observed in the inside of a stroma-rich primary pancreatic cancer organoid. The distinct network structure of HUVECs is confirmed within the primary pancreatic cancer organoid. In addition, hMSCs are confirmed to be present in the periphery of HUVECs so as to surround the HUVECs.

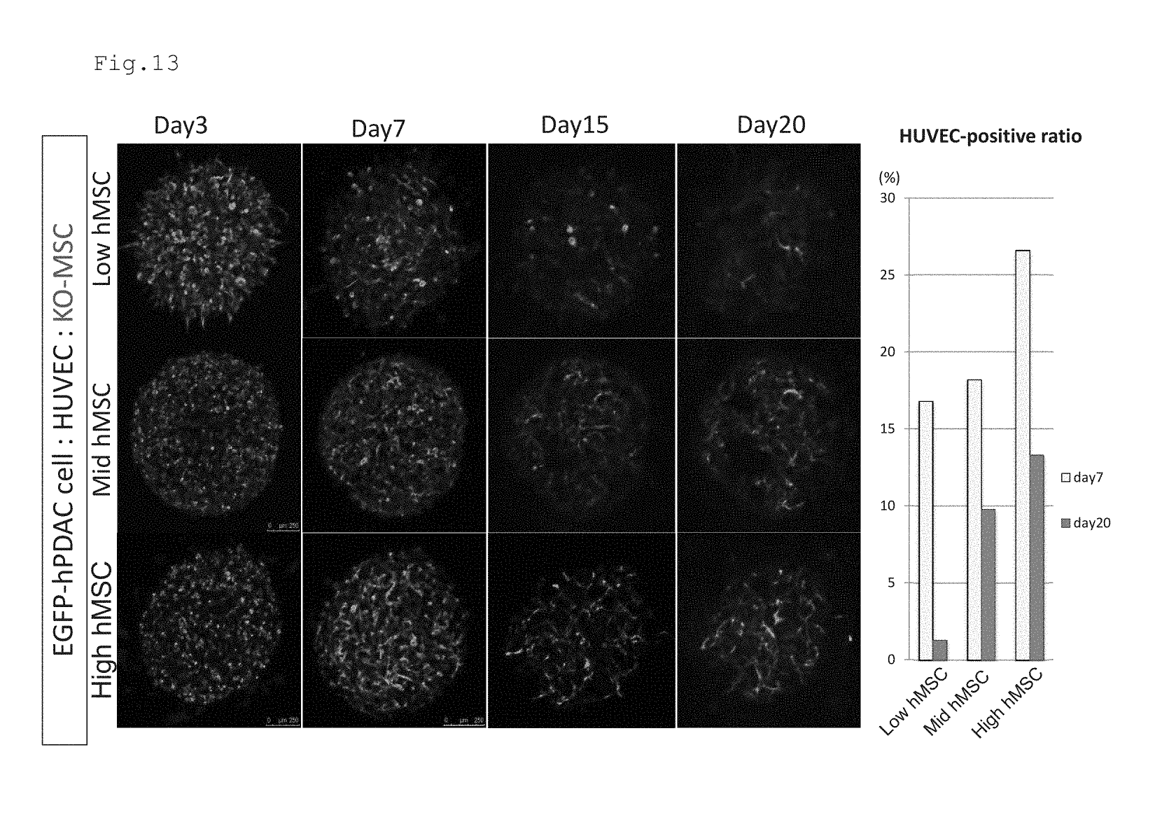

### 2-18 In Vitro Network Structure of Vascular Endothelial Cell Within Primary Human Pancreatic Cancer Organoid (FIG. 13)

Human primary pancreatic cancer cells (pancreatic cancer cells: 2×10⁵ cells), HUVECs, and hMSCs were three-dimensionally cocultured in vitro. A tissue image of the obtained primary pancreatic cancer organoid is shown. Used in this experiment were HUVECs transfected with GFP gene and hMSCs transfected with a gene encoding a red fluorescent protein (Kusabira Orange: KO). Abundant hMSCs promoted the network formation and maintenance of HUVECs.

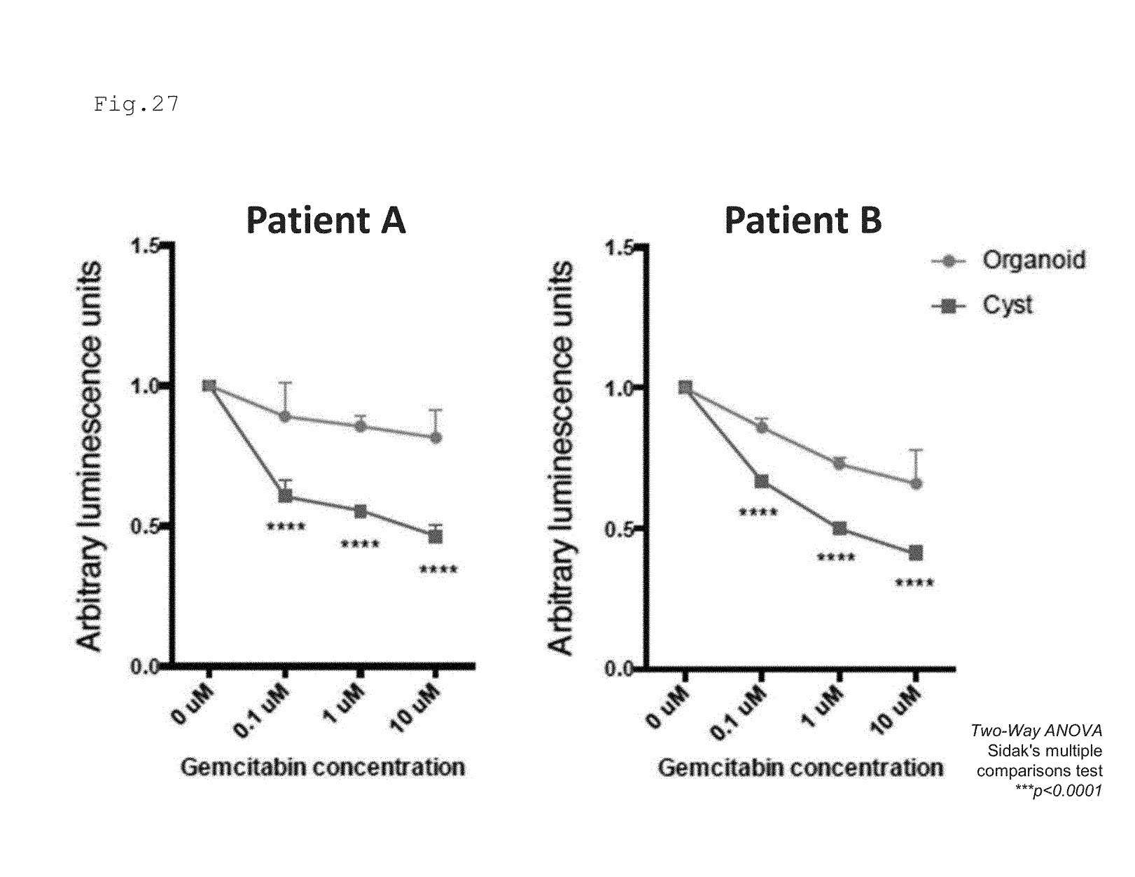

### 2-19 In Vitro Gemcitabine Sensitivity Evaluation of Primary Human Pancreatic Cancer Organoid (FIG. 27)

Luciferase gene-transfected primary human pancreatic cancer cells were established, and a primary pancreatic cancer organoid (pancreatic cancer cells: 2×10⁴ cells) was reconstituted in vitro and then cultured for 72 hours in the presence of gemcitabine. Then, a luminescent substrate was added thereto, and the luminescence intensity of each organoid was measured using a luminescence plate reader and analyzed. As a result of conducting statistical analysis (two-way ANOVA Sidak's multiple comparisons test), the primary pancreatic cancer organoid was confirmed to exhibit significantly high drug resistance as compared with pancreatic cancer cyst.

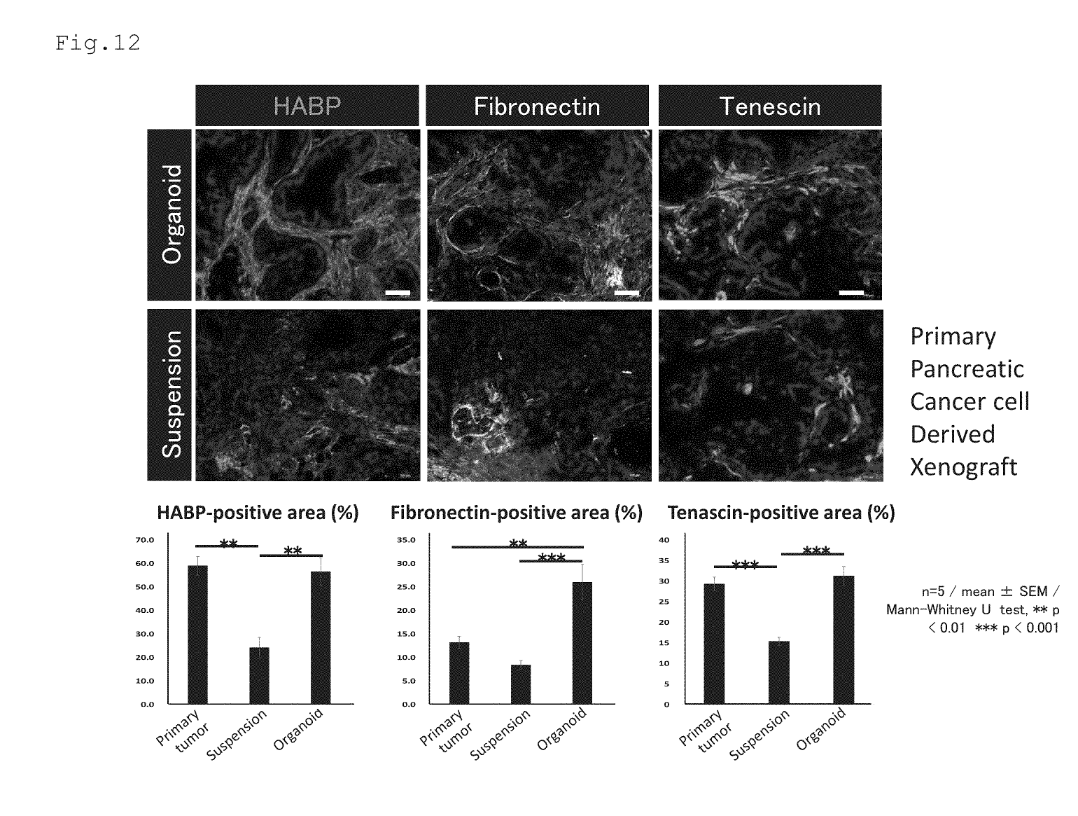

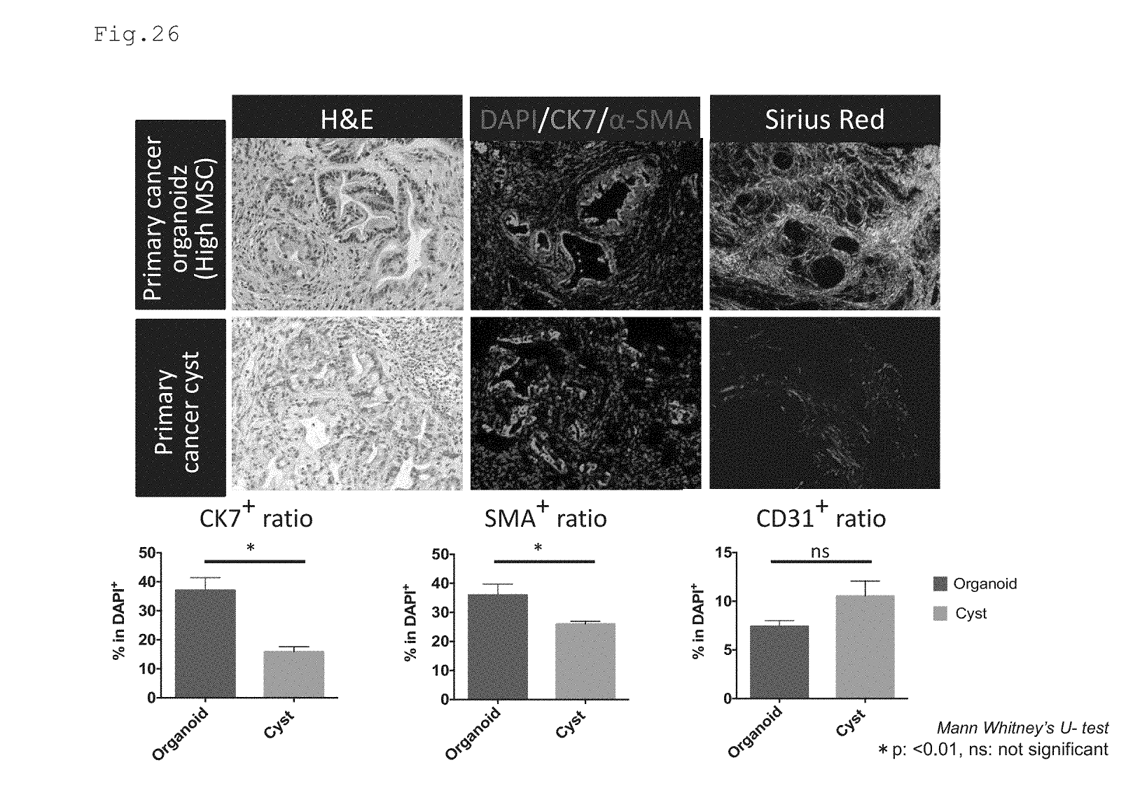

### 2-20 Enhanced Expression of Extracellular Matrices Characteristic of Pancreatic Cancer is Confirmed Within Human Primary Pancreatic Cancer Organoid-Derived Xenografts (FIGS. 26 and 12)

A primary pancreatic cancer organoid or primary pancreatic cancer cyst was reconstituted using human primary pancreatic cancer cells (pancreatic cancer cells: 2×10⁵ cells) and then transplanted to immunodeficient mice. An immunostaining image 1.5 months after the transplantation is shown. The upper panels show results for the primary pancreatic cancer organoid transplantation group, and the lower panels show results for the primary pancreatic cancer cyst transplantation group (FIG. 26). In contrast with the primary pancreatic cancer cyst transplantation group, a xenograft formed after primary pancreatic cancer organoid transplantation is confirmed to have a ductal structure characteristic of pancreatic cancer and, in addition, stroma constituted by αSMA-positive cells is detected. From a polarizing microscope image obtained after Sirius red staining, the xenograft formed after primary pancreatic cancer organoid transplantation is confirmed to be rich in collagen fiber in the transplanted region. FIG. 13 shows results of evaluating the expression of extracellular matrices in a xenograft formed after transplantation of a primary pancreatic cancer organoid or a primary pancreatic cancer suspension. This figure shows an immunostaining image of an extracellular matrix group including hyaluronic acid-binding protein (HABP), fibronectin, and tenascin. The enhanced expression of HABP, fibronectin, and tenascin is confirmed in the primary pancreatic cancer organoid transplantation group, and rich stroma is reconstituted within the primary pancreatic cancer organoid (FIG. 12).

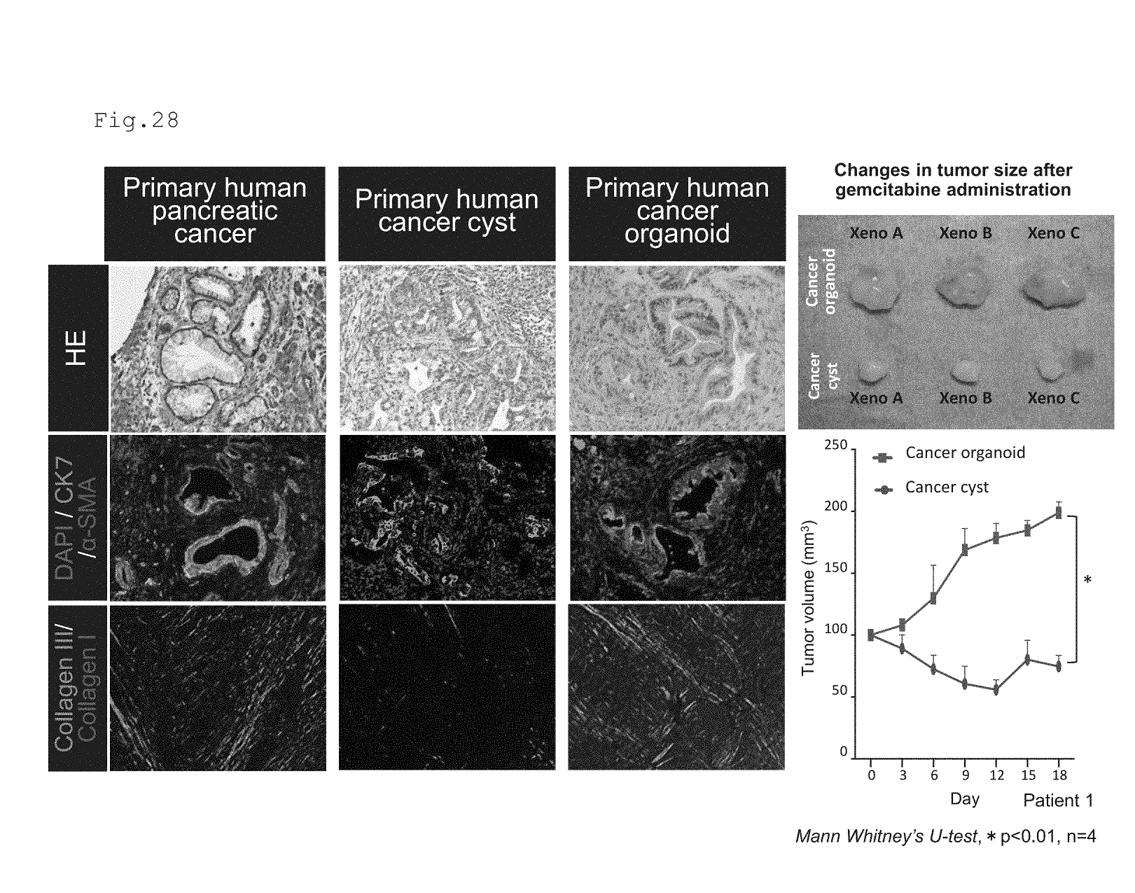

### 2-21 In Vivo Drug Sensitivity of Primary Human Pancreatic Cancer Organoid (FIG. 28)

A primary pancreatic cancer organoid (pancreatic cancer cells: 2×10⁵ cells) was reconstituted in vitro and then transplanted to immunodeficient mice. Subsequent variations in tumor size were observed. Gemcitabine was administered thereto once every three days from the point in time when a xenograft reached 100 mm³. The primary pancreatic cancer organoid transplantation group was confirmed to exhibit significantly high drug resistance as compared with a pancreatic cancer cyst transplantation group.

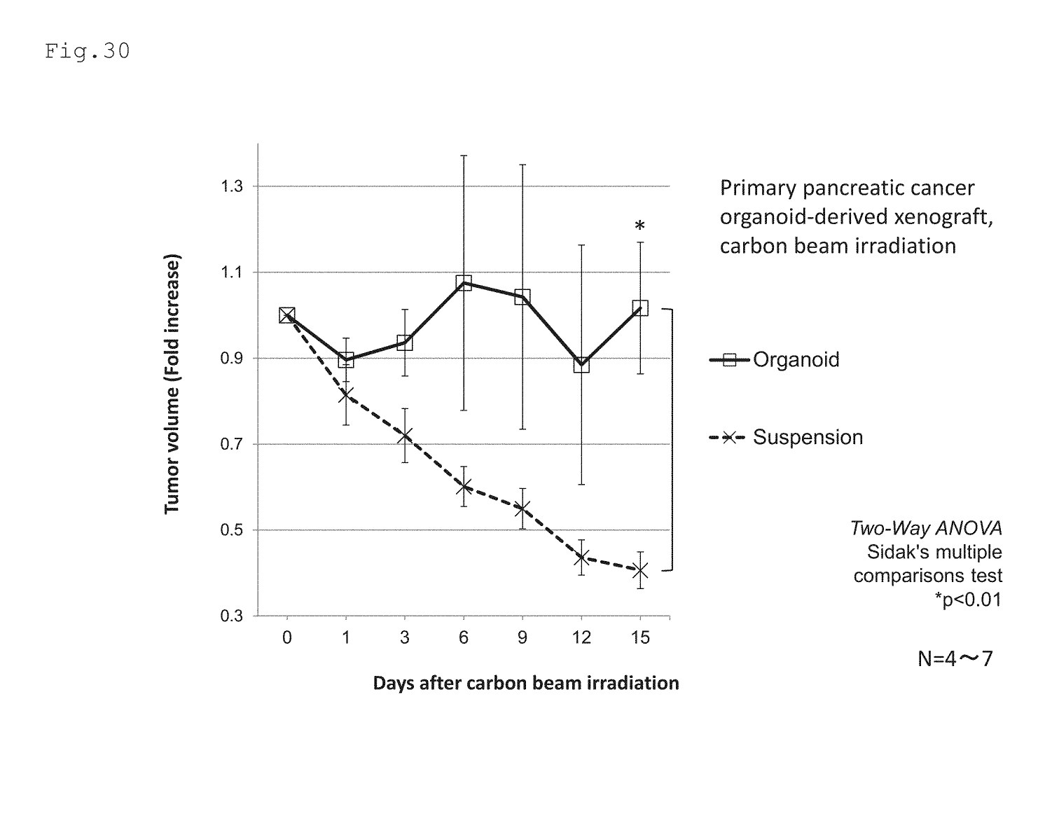

### 2-22 In Vivo Radiation Sensitivity of Human Primary Pancreatic Cancer Organoid (FIG. 30)

A primary pancreatic cancer organoid or a primary pancreatic cancer suspension was transplanted to immunodeficient mice, and after confirmation of tumor formation, the mice were exposed to radiation (carbon beam). Changes in tumor volume after the irradiation are shown. A marked decrease in tumor volume after the exposure to radiation is noted in the primary pancreatic cancer suspension transplantation group. On the other hand, the decrease in tumor volume after the irradiation with radiation is small in the primary pancreatic cancer organoid transplantation group.

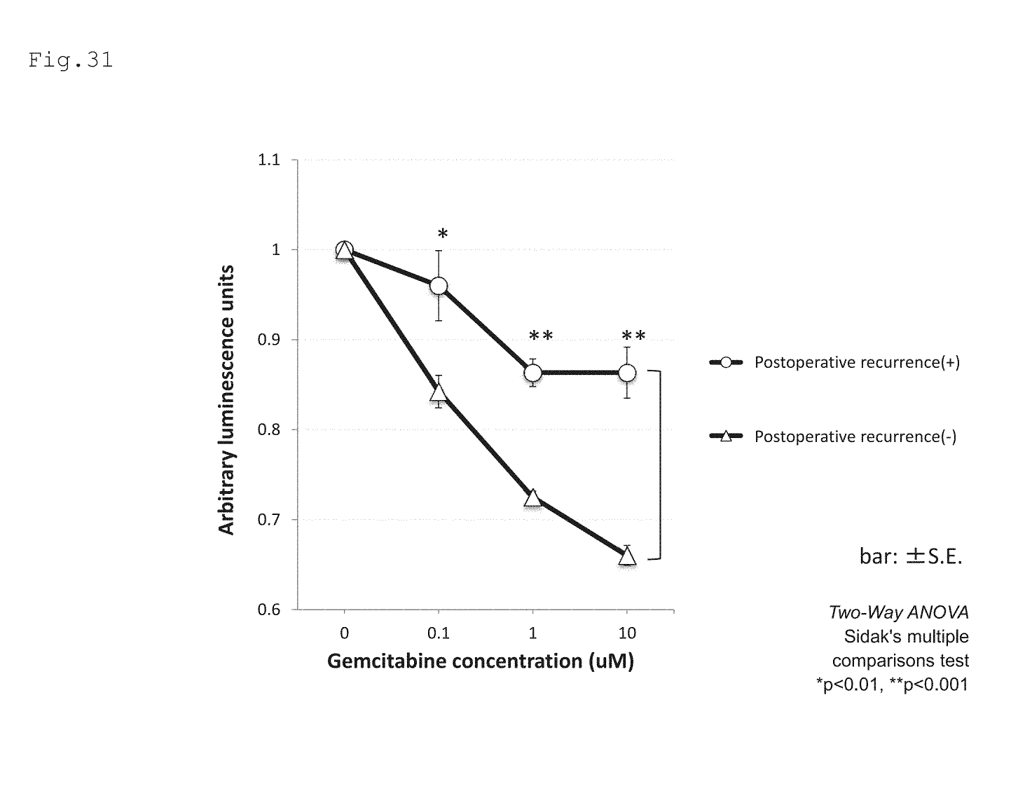

2-23 Correlation of the Drug Sensitivity of Primary Human Pancreatic Cancer Organoid with Patient Prognosis (FIG. 31)

Primary pancreatic cancer cells were separated from a surgically resected specimen of each pancreatic cancer patient (with or without postoperative recurrence), subjected to expanded culture, and then transfected with luciferase gene. Then, these cancer cells were three-dimensionally cocultured with stromal cells to reconstitute a primary pancreatic cancer organoid. The reconstituted human pancreatic cancer organoid was cultured for 72 hours in the presence of gemcitabine at respective concentrations, followed by luciferase activity measurement. The pancreatic cancer organoid derived from the surgically resected specimen of the lung cancer patient without postoperative recurrence exhibits sensitivity for gemcitabine, whereas the pancreatic cancer organoid derived from the surgically resected specimen of the pancreatic cancer patient having postoperative recurrence exhibits resistance to gemcitabine. On the other hand, a pancreatic cancer organoid derived from a surgically resected specimen of a pancreatic cancer patient having postoperative distant metastasis exhibits sensitivity for gemcitabine.

### INDUSTRIAL APPLICABILITY

The present invention is applicable as a tool for the evaluation of therapeutic resistance, such as in vivo drug sensitivity and radiation sensitivity, in drug discovery, the evaluation of therapeutic resistance, such as in vitro drug sensitivity and radiation sensitivity, in drug discovery, and for the elucidation of a mechanism underlying the treatment resistance of intractable cancer.

1. A reconstituted cancer organoid reproducing a cancer microenvironment.

2. The cancer organoid according to claim 1, wherein the cancer microenvironment comprises cancer stroma.

3. The cancer organoid according to claim 1, wherein the cancer organoid comprises cancer cells having the properties of epithelial cells.

4. The cancer organoid according to claim 1 further reproducing a ductal structure.

5. A reconstituted cancer organoid reproducing at least one selected from the group consisting of treatment resistance, invasion or metastasis, and recurrence of cancer.

6. The cancer organoid according to claim 5, wherein the treatment resistance of cancer is at least one selected from the group consisting of drug sensitivity, radiation sensitivity, immunotherapy sensitivity, and nutrition therapy sensitivity.

7. A reconstituted cancer organoid allowing prognostic prediction of cancer.

8. A method for preparing a cancer organoid, comprising: digesting cancer tissue in the presence of a proteolytic enzyme and a Rho kinase inhibitor and then obtaining an aggregate of cancer cells; passaging the aggregate and then separating the cancer cells; and
coculturing the cancer cells with mesenchymal cells and vascular endothelial cells to form the cancer organoid.

9. The method according to claim 8, wherein the cancer organoid reproduces a cancer microenvironment.

10. The method according to claim 9, wherein the cancer microenvironment comprises cancer stroma.

11. The method according to claim 8, wherein the cancer organoid comprises cancer cells having the properties of epithelial cells.

12. The method according to claim 8, wherein the cancer organoid further reproduces a ductal structure.

13. The method according to claim 8, wherein the cancer organoid reproduces at least one selected from the group consisting of treatment resistance, invasion or metastasis, and recurrence of cancer.

14. The method according to claim 13, wherein the treatment resistance of cancer is at least one selected from the group consisting of drug sensitivity, radiation sensitivity, immunotherapy sensitivity, and nutrition therapy sensitivity.

15. The method according to claim 8, wherein the cancer organoid allows prognostic prediction of cancer.

16. A method for preparing a xenograft reproducing a cancer microenvironment, comprising transplanting a nonhuman animal with a reconstituted cancer organoid reproducing a cancer microenvironment.

17. The method according to claim 16, wherein the cancer microenvironment of the xenograft comprises cancer stroma.

18. The method according to claim 16, wherein the reconstituted cancer organoid comprises cancer cells having the properties of epithelial cells.

19. The method according to claim 16, wherein the reconstituted cancer organoid further reproduces a ductal structure.

20. The method according to claim 16, wherein the xenograft further reproduces a ductal structure.

21. The method according to claim 16, wherein the xenograft reproduces at least one selected from the group consisting of treatment resistance, invasion or metastasis, and recurrence of cancer.

22. The method according to claim 21, wherein the treatment resistance of cancer is at least one selected from the group consisting of drug sensitivity, radiation sensitivity, immunotherapy sensitivity, and nutrition therapy sensitivity.

23. The method according to claim 16, wherein the xenograft allows prognostic prediction of cancer.

24. A xenograft reproducing a cancer microenvironment, the xenograft being obtained by transplanting a nonhuman animal with a reconstituted cancer organoid reproducing a cancer microenvironment.

25. The xenograft according to claim 24, wherein the cancer microenvironment of the xenograft comprises cancer stroma.

26. The xenograft according to claim 24, wherein the xenograft comprises cancer cells having the properties of epithelial cells.

27. The xenograft according to claim 24 further reproducing a ductal structure.

28. A reconstituted cancer organoid-derived xenograft reproducing at least one selected from the group consisting of treatment resistance, invasion or metastasis, and recurrence of cancer.

29. The xenograft according to claim 28, wherein the treatment resistance of cancer is at least one selected from the group consisting of drug sensitivity, radiation sensitivity, immunotherapy sensitivity, and nutrition therapy sensitivity.

30. A reconstituted cancer organoid-derived xenograft allowing prognostic prediction of cancer.

31. A reconstituted cancer organoid-derived xenograft reproducing expression of a drug transporter.

32. A reconstituted cancer organoid-derived xenograft having tumor vessels.

33. A reconstituted cancer organoid-derived xenograft reproducing drug leakage characteristic of tumor vessels.

34. A method for evaluating treatment resistance of cancer using a cancer organoid according to claim 1.

35. A method for evaluating invasion or metastasis of cancer using a cancer organoid according to claim 1.

36. A method for evaluating recurrence of cancer using a cancer organoid according to claim 1.

37. A method for conducting prognostic prediction of cancer using a cancer organoid according to claim 1.

38. A nonhuman animal carrying a xenograft according to claim 24.
